(19) United States
(12) Patent Application Publication
(10) Pub. No.: US 2023/0033358 A1
(43) Pub. Date: Feb. 2, 2023

(54) IMPLANTABLE MEDICAL DEVICE

(71) Applicant: Medtronic, Inc., Minneapolis, MN (US)

(72) Inventors: Kaileigh E. Rock, Mohtomedi, MN (US); Matthew D. Bonner, Plymouth, MN (US); Thomas A. Anderson, New Hope, MN (US)

(21) Appl. No.: 17/816,605

(22) Filed: Aug. 1, 2022

Related U.S. Application Data
(60) Provisional application No. 63/228,599, filed on Aug. 2, 2021.

(51) Int. Cl. A61N 1/05; A61N 1/375

(52) U.S. Cl. CPC A61N 1/05; A61N 1/3756; A61N 1/372

(57) ABSTRACT

An implantable medical device (IMD) including a fixation mechanism and a leadlet supporting an electrode. The leadlet includes a shape memory material configured to urge a leadlet body of the leadlet toward a preset orientation relative to a device body of the IMD. The leadlet is configured to establish a radial displacement between the device body and a distal end of the leadlet when the shape memory material urges the leadlet toward the preset orientation. The leadlet may be configured to cause the electrode to contact tissues of the heart when the fixation mechanism attaches to tissue of the heart and the shape memory material urges the leadlet toward the preset orientation.

This application claims the benefit of U.S. Provisional Application Ser. No. 63/228,599 (filed Aug. 2, 2021), which is entitled “IMPLANTABLE MEDICAL DEVICE” and is incorporated by reference herein in its entirety.

### TECHNICAL FIELD

This disclosure is related to an implantable medical systems, such as an implantable medical device.

### BACKGROUND

Implantable medical devices are often placed in a subcutaneous pocket and coupled to one or more transvenous medical electrical leads carrying pacing and sensing electrodes positioned in the heart. Intracardiac pacemakers have recently been introduced that are implantable within a ventricular chamber of a patient's heart for delivering ventricular pacing pulses without the use of electrical leads. Such pacemakers or other implantable medical devices may also be able to detect the occurrence of arrhythmias, such as fibrillation, tachycardia and bradycardia, in the patient's heart. An implantable cardiac defibrillator may deliver electrical shocks to the patient's heart in response to detection of a tachycardia or fibrillation to restore a normal heartbeat in the patient. In some cases, a single implantable medical device functions as both an implantable pacemaker and implantable cardiac defibrillator.

Implantable medical devices may include electrodes and/or other elements for physiological sensing and/or therapy delivery. The electrodes and/or other elements may be implanted at target locations selected to detect a physiological condition of the patient and/or deliver one or more therapies. For example, the electrodes and/or other elements may be delivered to a target location within an atrium or ventricle to sense intrinsic cardiac signals and deliver pacing or antitachyarrhythmia shock therapy from a medical device coupled to a lead.

### SUMMARY

This disclosure describes an implantable medical device (IMD) configured to position within a heart of a patient. The IMD includes a leadlet comprising a shape memory material configured to cause the leadlet to position an electrode of the leadlet in contact with tissue of the heart when a fixation mechanism secures the IMD to the heart. The shape memory material is configured to urge the leadlet to establish a substantially preset orientation relative to the device body, such that when a fixation mechanism of the IMD secures to the tissue, the leadlet radially displaces the electrode away from the device body.

In an example, a medical device comprises: a device body configured to position within a heart, the device body defining a device proximal end and a device distal end, and the device defining a longitudinal axis extending between the device proximal end and the device distal end; a fixation mechanism attached to a device distal end, wherein the fixation mechanism is configured to attach to tissue of the heart; and a leadlet mechanically supporting an electrode, wherein the leadlet defines a leadlet proximal end, a leadlet distal end, and a leadlet body between the leadlet proximal end and the leadlet distal end, wherein the leadlet proximal end is attached to the device body, wherein the leadlet body comprises a shape memory material configured to urge the leadlet body toward a preset orientation relative to the device body, and wherein the leadlet is configured to define a radial displacement between the leadlet distal end and the longitudinal axis, or an axis parallel to the longitudinal axis, when the shape memory material urges the leadlet body toward the preset orientation.

In an example, a medical device comprises: a device body configured to position within a heart, the device body defining a device proximal end and a device distal end, and the device defining a longitudinal axis extending between the device proximal end and the device distal end; a fixation mechanism attached to a device distal end, wherein the fixation mechanism is configured to attach to tissue of the heart; and a leadlet mechanically supporting an electrode, wherein the leadlet defines a leadlet proximal end, a leadlet distal end, and a leadlet body between the leadlet proximal end and the leadlet distal end, wherein the leadlet proximal end is attached to the device body, wherein the leadlet body comprises a shape memory material configured to urge the leadlet body toward a preset orientation relative to the device body, and wherein the leadlet is configured to define a radial displacement between the leadlet distal end and the longitudinal axis, or an axis parallel to the longitudinal axis, when the shape memory material urges the leadlet body toward the preset orientation, wherein the electrode is configured to contact a surface of the heart when the fixation mechanism attaches to the tissues of the heart and the shape memory material urges the leadlet body toward the preset orientation, and wherein the shape memory material is configured to generate an internal stress tending to oppose an external force exerted on the leadlet body when the shape memory material urges the leadlet body toward the preset orientation

In an example, a method comprises: establishing a radial displacement between a leadlet distal end of a leadlet and a longitudinal axis of a device body using a shape memory material configured to urge the leadlet body toward a preset orientation relative to the device body, wherein the leadlet body is between a leadlet proximal end and the leadlet distal end, wherein the leadlet proximal end is attached to the device body, and wherein the longitudinal axis extends between a device proximal end of the device body and a device distal end of the device body; and attaching a fixation mechanism to tissue of a heart, wherein the fixation mechanism is attached to the device distal end, wherein the device body is configured to position within the heart, and wherein the leadlet mechanically supports an electrode configured to contact a surface of the heart when the shape memory material urges the leadlet body toward the preset orientation.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical system including an implantable medical device.

FIG. 2 is a perspective view of an implantable medical device with an example leadlet substantially establishing a preset orientation.

FIG. 3 is a perspective view of an implantable medical device of with another example leadlet substantially establishing a preset orientation.

FIG. 4 is a plan view of an example leadlet defining a radial displacement from a longitudinal axis.

FIG. 5 is a plan view of another example leadlet defining a radial displacement from a longitudinal axis.

FIG. 6A is a schematic illustration of an implantable medical device within a delivery catheter.

FIG. 6B is a schematic illustration of the implantable medical device of FIG. 6B with the delivery catheter withdrawn.

FIG. 7 is a perspective view of an implantable medical device including a first example of an electrode.

FIG. 8 is a perspective view of an implantable medical device including a second example of an electrode.

FIG. 9 is a perspective view of an implantable medical device including a third example of an electrode.

FIG. 10 is a perspective view of an implantable medical device including a fourth example of an electrode.

FIG. 11 is a perspective view of an implantable medical device including a fifth example of an electrode.

FIG. 12 is a perspective view of an example leadlet extending proximally when the leadlet substantially establishing the preset orientation.

FIG. 13 is a perspective view of an implantable medical device including a first leadlet and second leadlet.

FIG. 14 is a flow diagram that illustrates an example technique for using the example implantable medical device.

### DETAILED DESCRIPTION

This disclosure describes an implantable medical device (IMD) configured to position an electrode of a leadlet in contact with tissue of a patient, such as a septal wall of the heart. The IMD is configured to position within a heart of a patient, such as within an atrium, ventricle, coronary sinus, or other portions of the heart. The leadlet is secured to a device body of the IMD and configured to cause contact between the electrode and the heart when the fixation mechanism secures the device body to the tissue.

The leadlet comprises a shape memory material configured to urge the leadlet to establish a substantially preset orientation relative to the device body, such that when a fixation mechanism of the IMD secures to the tissue, the leadlet radially displaces the electrode away from the device body. In examples, the preset orientation causes the leadlet to position the electrode in contact with the tissues of the heart at a location displaced from the attachments point(s) of the fixation mechanism. This may increase the available locations for an electrode to position when, for example, space constraints limit the areas where a fixation mechanism may attach, when it may be advantageous for the electrode to substantially avoid an attachment point of the fixation mechanism, and/or for other reasons.

The preset orientation of the leadlet may be defined by a geometric description of some portion of the leadlet expressed relative to the device body. For example, the preset orientation may be defined by a general shape of the leadlet relative to the device body, a curvature of the leadlet relative to the device body, a position of some portion of the leadlet relative to the device body, or some other geometric description. The shape memory material is configured to cause the leadlet to establish the preset orientation in the absence of external forces acting on the leadlet (e.g., an external force tending to cause the geometric description of the leadlet to depart from the preset orientation).

In examples, the shape memory material is resiliently biased to cause the preset orientation such that when an external force causes the leadlet to depart from the preset orientation, the shape memory material generates forces within the leadlet tending to urge the leadlet toward the preset orientation (e.g., to urge the leadlet to reestablish the preset orientation). For example, when the fixation mechanism of the device secure to tissues of the heart and the preset orientation causes the leadlet and/or the leadlet-supported electrode to contact a surface of the heart and slightly depart from the preset orientation, the shape memory material may urge the leadlet toward the preset orientation (e.g., to urge the leadlet to reestablish the preset orientation), causing the leadlet and/or the leadlet-supported electrode to substantially maintain contact with the surface of the heart. In other examples, the leadlet may be configured such that, when the fixation mechanism of the device secure to tissues of the heart, the preset orientation causes the leadlet and/or the leadlet-supported electrode to contact the surface of the heart without departing from the preset orientation.

The leadlet includes a leadlet body mechanically supporting the electrode. The leadlet body includes a proximal end (“leadlet proximal end”) mechanically coupled (e.g., attached) to the device body and a distal end (“leadlet distal end”) opposite the proximal end. The leadlet body is configured such that, when the shape memory material causes the leadlet to substantially establish the preset orientation, the leadlet body defines a radial displacement between the leadlet distal end and a longitudinal axis defined by the device body, and/or between the leadlet distal end and an axis parallel to the longitudinal axis. The leadlet body may be configured such that, when the shape memory material urges the leadlet toward the preset orientation, the leadlet body defines some fraction of the radial displacement. The radial displacement may cause the leadlet body to position the electrode at a location displaced from the longitudinal axis of the device body, and/or displaced from the axis parallel to the longitudinal axis. In examples, the device body defines a proximal end (“device proximal end”) and a distal end (“device distal end”) opposite the device proximal end, and the longitudinal axis extends from the device proximal end to the device distal end. The IMD includes a fixation member configured to secure the device body (e.g., the device distal end) distal end to tissues of the heart. The leadlet proximal end may be mechanically coupled to the device distal end, a portion of the device body between the device distal end and the device proximal end, or the device proximal end.

The IMD may be configured such that when the fixation mechanism secures the device body to tissues of the heart, the preset orientation of the leadlet tends to place an electrode mechanically supported by the leadlet in contact with the tissues of the heart at a location displaced from the attachment point(s) of the fixation mechanism. The preset orientation of the leadlet body may cause the leadlet body to displace the electrode in a direction away from the device body. The preset orientation may cause the leadlet body to substantially maintain the electrode in a defined position relative to the device body. The defined position may be, for example, distal to the device distal end and radially displaced from the longitudinal axis of the device body, such that when the fixation mechanism secures the device distal end to tissues of the heart, the leadlet body may cause the electrode to contact the tissues based on the position defined by the preset orientation. The leadlet body may be configured such that the preset orientation defines other positions of the electrode relative to the device body in other examples.

In examples, the electrode is a contact electrode configured to contact a surface of the heart. The leadlet may be configured to place the electrode in contact with a surface of the heart when the shape memory material urges the leadlet to substantially establish the preset orientation and/or urges the leadlet toward the preset orientation. In examples, the leadlet is configured such that the preset orientation causes the leadlet body to substantially maintain the electrode in contact with a heart surface when the fixation mechanism of the device body attaches to the tissue of the heart. The leadlet may be configured such that the preset orientation causes some portion of the leadlet body to substantially lay atop the heart surface when the fixation mechanism attaches to the tissue of the heart, such that the leadlet substantially maintains the electrode in contact with the heart surface during movement of the heart (e.g., a heart beat).

In examples, the preset orientation of the leadlet body causes the leadlet body to define a curvature between the leadlet proximal end and the leadlet distal end. In examples, the leadlet body defines a positive curvature causing the leadlet body to curve away from the longitudinal axis of the device body. The curvature may defined to cause a portion of the leadlet body mechanically supporting the electrode to contact a surface of the heart when the fixation mechanism is attached to the tissues of the heart and the leadlet body substantially establishes the preset orientation. In some examples, the leadlet body includes an angled portion defining an angle between the leadlet proximal end and a portion of the leadlet mechanically supporting the electrode, and the angled portion is configured to cause the portion mechanically supporting the electrode to contact a surface of the heart when the fixation mechanism is attaches to the tissues of the heart.

In some examples, the leadlet body is an elongated body extending between the leadlet proximal end and the leadlet distal end. The elongate body may define a circumference substantially perpendicular to a leadlet axis extending through the leadlet body from the leadlet proximal end to the leadlet distal end. The leadlet axis may define curved sections and/or linear sections. In examples, the circumference of the elongated body is substantially ovalur (e.g., oval shaped or substantially circular), polygonal, or a shape having both straight sides and curved sides. In some examples, the leadlet body substantially defines a sheet defining a first side and a second side opposite the first side, with the first side defining a substantially planar first surface and the second side and defining a substantially planar second surface. The leadlet body may be configured to position the first surface facing toward the surface of the heart when fixation mechanism attaches to the heart and the leadlet body substantially establishes the preset orientation. The leadlet body may be configured to position the first surface facing toward the surface of the heart when fixation mechanism attaches to the heart and the shape memory material urges the leadlet to substantially establish the preset orientation and/or urges the leadlet toward the preset orientation. In examples, the electrode is configured to contact the surface of the heart when the first surface contacts the tissue of the heart.

The shape memory material may be any material capable of being resiliently biased. In examples, the shape memory material comprises a shape memory polymer and/or a shape memory alloy. The shape memory material may be configured such that its resilient biasing tends to cause the leadlet body to assume a given shape in the absence of external forces exerted on the leadlet body. The shape memory material may be configured such that, when the leadlet distal end or some other portion of the leadlet body is displaced, the resilient biasing of the shape memory material causes the leadlet body to generate internal stresses tending to resist the displacement. Hence, the leadlet body may be configured such that the shape memory alloy positions the electrode in a substantially consistent position away from and relative to the device body (e.g., in contact with a heart surface) when the fixation mechanism secures the device body to the tissue of the heart.

The IMD may include circuitry configured to deliver therapy signals to and/or sense signals of the heart of the patient using the electrode. The leadlet body may mechanically support a conductor electrically connected to the electrode. In examples, the conductor is electrically connected to circuitry mechanically supported by the device body (e.g., in a hermetically sealed enclosure defined by the device body). The leadlet body may mechanically support a plurality of electrodes, with the leadlet body configured to cause one or more of the plurality of electrodes to contact the tissues of the heart when the shape memory material urges the leadlet to substantially establish the preset orientation and/or urges the leadlet toward the preset orientation. The conductor may be electrically connected to two or more electrodes in the plurality of electrodes. In some examples, the plurality of electrodes includes at least a first electrode and a second electrode, and the leadlet body mechanically supports a first conductor electrically connected to the first electrode and a second conductor electrically connected to the second electrode. The leadlet body may be configured to electrically insulate the first conductor from the second conductor, and vice-versa, such that, for example, the circuitry may deliver and/or sense a first signal using the first electrode and first conductor and deliver and/or sense a second signal using the second electrode and second conductor.

In some examples, the fixation mechanism includes one or more tines extending from the device distal end and configured to penetrate the tissues of the heart to secure the device body to the tissues of the heart. A tine may be any elongated body having any shape configured to penetrate tissue of the heart to substantially attach the IMD to the heart. In examples, a tine includes a fixed end mechanically coupled to the device body (e.g., the device distal end) and a free end opposite the fixed end. The free end may be configured to penetrate the tissues of the heart. In examples, the tine is biased to drive the free end radially outward from the longitudinal axis of the device body to cause the tine to secure the device body to the tissues of the heart. In examples, the one or more tines include a first tine and a second tine. The leadlet body may be configured such that the leadlet proximal end is attached to device distal end and the leadlet body passes between the first tine and the second tine when the shape memory material urges the leadlet to substantially establish the preset orientation and/or urges the leadlet toward the preset orientation. In some examples, the tine has a helical shape (e.g., defining a helix). Hence, the leadlet body may be configured to place the electrode in contact with the tissues of the heart at a location displaced from the attachments points of the fixation mechanism.

Thus, the IMD may be configured to position the electrode of the leadlet in a position radially displaced from the device body when the fixation mechanism secures to tissues of a patient, such as a septal wall of the heart. The leadlet may be configured such that the shape memory material of the leadlet body causes the leadlet to establish a substantially preset orientation relative to the device body to cause the leadlet to radially displace the electrode away from the device body. The leadlet may be configured to position the electrode such that electrical contact between the electrode and the tissues of the heart occurs at a location displaced from the attachments point(s) of the fixation mechanism to, for example, increase the positioning flexibility for the electrode when space constraints limit available attachment locations for the fixation mechanism, cause the electrode to substantially avoid an attachment point of the fixation mechanism, and/or for other reasons.

The example systems, devices, and techniques of this disclosure will be described with reference to delivering electrodes of an IMD configured as a cardiac pacemaker to a target site in the heart of a patient. However, the example systems, devices, and techniques are not limited to delivering such IMD electrodes to target sites within a heart. For example, the example systems, devices, and techniques described herein may be used to deliver other medical devices, such as sensing devices, neurostimulation device, medical electrical leads, etc. Additionally, the example systems, devices, and techniques described herein may be used to deliver any such IMDs to other locations within the body of the patient. The example systems, devices, and techniques described herein can find useful application in delivery of a wide variety of implantable medical devices for delivery of therapy to a patient or patient sensing.

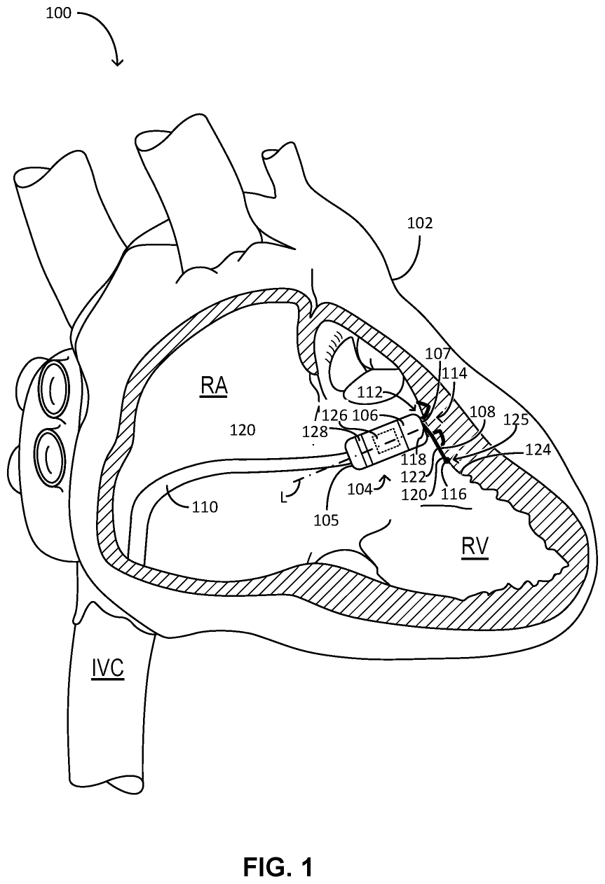

FIG. 1 is a conceptual diagram illustrating a portion an example medical system 100 configured to deliver therapy (e.g., pacing) to a heart 102 of a patient. Medical system 100 includes IMD 104 including device body 106 and leadlet 108 extending from device body 106. Medical system 100 includes a delivery catheter 110 configured to position IMD 104 within the vicinity of a target site 112 within heart 102. In examples, as illustrated in FIG. 1, target site 112 is a region in a ventricular wall of the right ventricle (RV) of heart 102. In other examples, delivery catheter 110 and/or IMD 104 may be configured to position in the vicinity of a target site at another portion of heart 102. For example, delivery catheter 110 and/or IMD 104 implantable medical lead may be configured to position in the vicinity of a target site in the right atrium (RA) of heart 102, the left atrium of heart 102 (not shown), or the left ventricle of heart 102 (not shown). Delivery catheter 110 and IMD 104 may be configured to extend through vasculature of a patient (e.g., an interior vena cava (IVC)) to position IMD 104 within heart 102. In examples, delivery catheter 110 includes a cup section (not shown) defining a lumen configured to engage IMD 104.

IMD 104 includes a fixation mechanism 114 configured to secure IMD 104 (e.g., device body 106) to tissues of heart 102. In examples, device body 106 mechanically supports fixation mechanism 114. Fixation mechanism 114 is configured to penetrate tissue of heart 102 at or near a target site, such as target site 112. For example, fixation mechanism 114 may be configured to penetrate cardiac tissue of a septal wall in a RV, RA, LV, and/or LA of heart 102, or penetrate cardiac tissue in another area of heart 102. Fixation mechanism 114 may be configured to substantially maintain IMD 104 at or in the vicinity of the target site when fixation mechanism 114 penetrates tissues at or in the vicinity of the target site. In examples, device body 106 defines a proximal end 105 (“device proximal end 105”) and a distal end 107 (“device distal end 107”) opposite device proximal end 105. Device body 106 may mechanically support fixation mechanism 114 substantially at device distal end 107. In some examples, fixation mechanism 114 may include one or more tines (e.g., a helical tine) configured as electrodes for pacing and sensing in the implant chamber, e.g., atrial pacing and sensing.

Fixation mechanism 114 may be configured to allow a clinician to cause fixation mechanism 114 to engage the tissue within heart 102, such that the clinician may affix IMD 104 once delivered to the target site. For example, fixation mechanism 114 may include one or more tines configured to position within the cup section of delivery catheter 110 when IMD 104 is positioned within the cup section, with the one or more tines resiliently biased to deploy outward to grasp tissue when delivery catheter 110 is proximally withdrawn (e.g., by the clinician). In some examples, fixation mechanism 114 may include a helical element, a barbed element, screws, rings, and/or other structures configured to resist a translation (e.g., a proximal translation) of device body 106 away from a tissue wall when fixation mechanism 114 is engaged with the tissue wall. Hence, medical system 100 may be configured such that a clinician may guide IMD 104 to the vicinity of a target site such as target site 112 using delivery catheter 110, then cause fixation mechanism 114 to substantially maintain IMD 104 at or in the vicinity of the target site.

Leadlet 108 mechanically supports an electrode 116. Leadlet 108 is configured such that, when fixation mechanism 114 causes IMD 104 to attach to tissues of heart 102, leadlet 108 defines a radial displacement between electrode 116 and longitudinal axis L, and/or defines a radial displacement between electrode 116 and an axis parallel to longitudinal axis L. In examples, the radial displacement between electrode 116 and longitudinal axis L is substantially perpendicular to longitudinal axis L. In examples, the radial displacement between electrode 116 and the axis parallel to longitudinal axis L is substantially perpendicular to longitudinal axis L. Leadlet 108 may comprise a shape memory material such as a shape memory polymer and/or shape memory alloy configured to cause leadlet 108 to define the radial displacement.

In examples, the shape memory material is resiliently biased to cause leadlet 108 to substantially establish a preset orientation relative to device body 106. The shape memory material may be resiliently biased to urge leadlet 108 toward the preset orientation relative to device body 106. The preset orientation relative to device body 106 may cause leadlet 108 to define the radial displacement. IMD 104 may be configured such that the shape memory material causes leadlet 108 to generate contact between electrode 116 and a surface of heart 102 when fixation mechanism 114 causes IMD 104 to attach to tissues of heart 102. Hence, leadlet 108 may be configured to position electrode 116 such that electrical contact between electrode 116 and the tissues of the heart 102 occurs at a location displaced from the attachments point(s) of fixation mechanism 114 to, for example, increase the positioning flexibility for electrode 116 when space constraints limit available attachment locations for fixation mechanism 114, cause electrode 116 to substantially avoid (e.g., displace from) an attachment point of fixation mechanism 114, and/or for other reasons.

Leadlet 108 may include a proximal end 118 (“leadlet proximal end 118”), a distal end 120 (“leadlet distal end 120”) opposite leadlet proximal end 118, and a leadlet body 122 extending between and defining leadlet proximal end 118 and leadlet distal end 120. Leadlet proximal end 118 may be mechanically coupled (e.g., attached to) device body 106. In examples, leadlet proximal end 118 is mechanically coupled to device distal end 107. In some examples, leadlet proximal end 118 is mechanically coupled to a portion of device body 106 between device distal end 107 and device proximal end 105. In some examples, leadlet proximal end 118 is mechanically coupled to device proximal end 105. Leadlet body 122 may mechanically support electrode 116. In examples, leadlet body 122 mechanically supports electrode 116 on a distal portion of leadlet body 122, wherein the distal portion includes lead distal end 120.

The shape memory material of leadlet body 122 may be resiliently biased such that, when leadlet body 122 substantially establishes the preset orientation, the shape memory material generates an internal stress tending to oppose an external force on leadlet body 122 which causes leadlet body 122 to depart from the preset orientation. In examples, the internal stress acts to cause the shape memory material to cause leadlet body 122 to attempt to establish or reestablish the preset orientation when the external force causes the departure. For example, when fixation mechanism 114 attaches to heart 102 and the preset orientation causes leadlet body 122 to contact a heart surface 124 of heart 102, heart surface 124 may exert a force on leadlet body 122 tending to cause a slight departure from the preset orientation of leadlet body 122. In response, the shape memory material may generate an internal stress opposing the force of heart surface 124, such that leadlet body 122 substantially remains in contact with heart surface 124.

In examples, leadlet 108 is configured to cause electrode 116 to contact heart surface 124 when the shape memory material urges leadlet body 122 to substantially establish the preset orientation and/or urges leadlet body 122 toward the preset orientation. In some examples, leadlet body 122 defines a curvature between leadlet proximal end 118 and leadlet distal end 120 configured to cause leadlet body 122 and/or electrode 116 to contact heart surface 124 when fixation mechanism 114 is attached to the tissues of heart 102 and the shape memory material urges leadlet body 122 to substantially establish the preset orientation and/or urges leadlet body 122 toward the preset orientation. Hence, IMD 104 may be configured such that, as IMD 104 approaches target site 112, the preset orientation of leadlet body 122 may cause electrode 116 to contact heart surface 124. When fixation mechanism 114 engages tissues of heart 102, and heart surface 124 causes a slight departure of leadlet body 122 from the preset orientation, the shape memory material may urge leadlet body 122 toward heart surface 124 such that leadlet body 122 substantially maintains electrode 116 in contact with heart surface 124. For example, the shape memory material may urge leadlet body 122 toward heart surface 124 to cause leadlet body 122 to substantially maintain electrode 116 in contact with heart surface 124 at an electrode location 125 radially displaced from longitudinal axis L.

IMD 104 may include circuitry 126 configured to deliver therapy signals to and/or sense signals of heart 102 using electrode 116. Circuitry 126 may be configured to deliver electrical signals to cause the cardiac muscle, e.g., of the ventricles, to depolarize and, in turn, contract at a regular interval. Circuitry 126 may be mechanically supported by device body 106 (e.g., in a hermetically sealed enclosure defined by device body 106), although this is not required. In examples, leadlet body 122 mechanically supports a conductor (not shown) electrically connected to electrode 116. The conductor may be electrically connected to circuitry 126, such that circuitry 126 may electrically communicate with electrode 116. In examples, circuitry 126 may include one or more of sensing circuitry (e.g., for sensing cardiac signals), therapy delivery circuitry (e.g., for generating cardiac pacing pulses), and processing circuitry for controlling the functionality of IMD 104.

In some examples, leadlet body 122 may mechanically support a plurality of electrodes and/or a plurality of conductors, with each conductor electrically connected to at least one electrode. In some examples, leadlet body 122 is configured to electrically insulate a first conductor from a second conductor, and vice-versa, such that circuitry 126 may deliver and/or sense a first signal using the first electrode and first conductor and deliver and/or sense a second signal using the second electrode and second conductor. Device body 106 may define a return electrode 128 electrically connected to circuitry 126. Circuitry 126 may be configured to deliver therapy to and/or sense signals from heart 102 using return electrode 128. In some examples, device body 106 may mechanically support additional leads and/or electrodes configured to deliver therapy to and/or sense signals from heart 102. The additional leads and/or electrodes may be configured as contact electrodes configured to contact a surface of heart 102 and/or may be configured as penetrating electrodes configured to penetrate and implant in tissues of heart 102. In some examples, electrode 116 is a contact electrode configured to contact heart surface 124 when fixation mechanism 114 engages tissues of heart 102, and device body 106 includes a second leadlet mechanically supporting a second electrode configured to penetrate and implant within tissue of heart 102 when fixation mechanism 114 engages tissues of heart 102. The second electrode may be a deep penetrating electrode operably coupled to circuitry 126. In examples, circuitry 126 is configured to deliver therapy signals to and/or sense signals of heart 102 in a heart chamber in which IMD 104 (e.g., the RV or RA) using electrode 116, and configured to deliver therapy signals to and/or sense signals of heart 102 in cardiac tissue of another chamber and/or the conduction system of heart 102 using the second electrode.

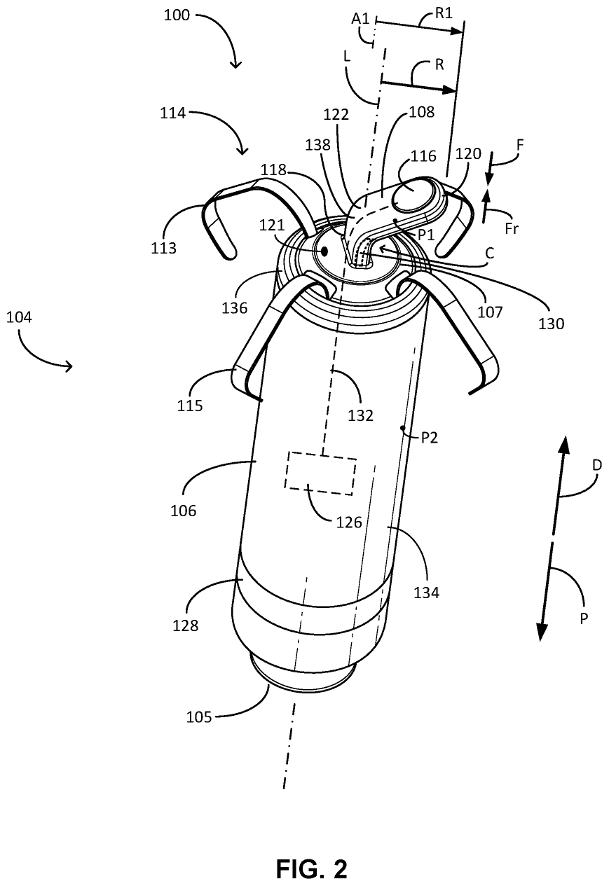

FIG. 2 illustrates medical system 100 including a perspective view of an example IMD 104. IMD 104 defines longitudinal axis L extending through device distal end 107 and device proximal end 105. Leadlet 108 defines a preset orientation with respect to device body 106. Leadlet 108 defines a radial displacement R between leadlet distal end 120 and longitudinal axis L when leadlet 108 substantially establishes the preset orientation. Leadlet 108 defines a radial displacement R1 between leadlet distal end 120 and an axis A1 parallel to longitudinal axis L when leadlet 108 substantially establishes the preset orientation. In examples, the radial displacement R and/or the radial displacement R1 is substantially perpendicular to longitudinal axis L. Leadlet body 122 comprises a shape memory material 130 (shown in dashed lines) configured to cause leadlet body 122 to substantially establish the preset orientation with respect to device body 106. In examples, shape memory material 130 is configured to urge leadlet body 122 toward the preset orientation when leadlet body 122 departs from the preset orientation.

FIG. 2 illustrates leadlet body 122 in a relaxed configuration. In the relaxed configuration depicted, leadlet body 122 is substantially free of forces external to IMD 104 acting on leadlet body 122, and any stresses on or within leadlet body 122 arise from properties or phenomena internal to leadlet body 122, such as mass, internal temperature, residual stresses, and the like. In the relaxed configuration, shape memory material 130 causes leadlet body 122 to substantially establish the preset orientation whereby leadlet 108 defines the radial displacement R between leadlet distal end 120 and longitudinal axis L.

Shape memory material 130 may be resiliently biased such that, when leadlet body 122 substantially establishes the preset orientation and/or shape memory material 130 urges leadlet body 122 toward the preset orientation, shape memory material 130 tends to oppose an external force on leadlet body 122 which causes leadlet body 122 to depart from the preset orientation. In examples, shape memory material 130 is configured to generate an internal stress tending to cause leadlet body 122 to attempt to establish or reestablish the preset orientation when the external force causes a departure from the preset orientation. Although illustrated in FIG. 2 in the vicinity of leadlet proximal end 118 and device distal end 107 for clarity, shape memory material 130 may be located in other places of IMD 104 in other examples. In some examples, shape memory material 130 may comprise a portion of leadlet body 122 substantially from leadlet proximal end 105 to leadlet distal end 107, such that shape memory material 130 extends over the length of leadlet body 122. In some examples, shape memory material 130 may define and/or be substantially inseparable from leadlet body 122. For example, shape memory material 130 may be a shape memory polymer or metal treated and/or fabricated to exhibit a resilient bias and defining substantially the entirety of leadlet body 122.

The resilient biasing of shape memory material 130 results in a tendency of leadlet body 122 to return or attempt to return to an initial position defined by the preset orientation when leadlet body 122 is temporarily displaced (e.g., departs from) from the initial position. For example, the preset orientation may define an initial position of a point P1 on leadlet body 122 relative to a point P2 on device body 106. Shape memory material 130 may be configured such that, when a force F acts on the leadlet body 122 to displace point P1 proximally (e.g., in the proximal direction P) from the initial position, shape memory material 130 urges leadlet body 122 to return or attempt to return the point P1 to the initial position by causing leadlet body 122 to exert a reaction force Fr opposing the force F (e.g., in the distal direction D). In examples, when fixation mechanism 114 attaches to heart 102 and contact with heart surface 124 causes a slight departure from the preset orientation of leadlet body 122, shape memory material 130 urges leadlet body 122 against heart surface 124 such that leadlet body 122 and/or electrode 116 substantially remain in contact with heart surface 124. As discussed, in other examples, when fixation mechanism 114 attaches to heart 102, the preset orientation may cause leadlet body 122 and/or electrode 116 to contact heart surface 124 without substantially departing from the preset orientation.

Leadlet body 122 may mechanically support electrode 116 such that leadlet body 122 causes a radial displacement between electrode 116 and longitudinal axis L when leadlet body 122 substantially establishes the preset orientation and/or is urged toward the preset orientation. Leadlet body 122 may cause the radial displacement between electrode 116 and longitudinal axis L when leadlet body 122 defines the radial displacement R. In examples, leadlet 108 is configured such that, when fixation mechanism 114 attaches to a tissue wall within target site 112 of heart 102 (FIG. 1), contact between the tissue wall and leadlet body 122 causes leadlet body 122 to substantially flatten (e.g., move proximally toward device distal end 107). Leadlet body 122 may be configured such that the movement toward device distal end 107 increases the radial displacement R and increases the radial displacement R between electrode 116 and longitudinal axis L and/or increases the radial displacement R1 between electrode 116 and axis A1. For example, leadlet body 122 may be configured such that the attachment of fixation mechanism 114 to the tissue wall within target site 112 causes leadlet body 122 to substantially flatten and increase the radial displacement R and/or the radial displacement R1 to position electrode 116 substantially at or in the vicinity of electrode location 125 on heart surface 124. Leadlet 108 may be configured such that the substantial flattening of leadlet body 122 causes a departure from the preset orientation of leadlet body 122, causing shape memory material 130 to urge leadlet body 122 toward heart surface 124 to substantially maintain electrode 116 in contact with heart surface 124 as, for example, heart surface 124 moves during cardiac activity.

The preset orientation of leadlet body 122 caused by shape memory material 130 may cause leadlet body 122 to define a curvature C relative to longitudinal axis L of device body 106. Leadlet body 122 may define the curvature C substantially between leadlet proximal end 118 and leadlet distal end 120. In examples, leadlet body 122 is configured to define the curvature C such that when fixation mechanism 114 attaches to a tissue wall, the curvature C causes leadlet body 122 to extend toward the tissue wall. In examples, when fixation mechanism 114 attaches to the tissue wall, leadlet body 122 is configured such that the curvature C position electrode 116 to face and/or contact the tissue wall. In some examples, the curvature C causes leadlet body 122 to substantially curve away from longitudinal axis L. In examples, leadlet body 122 defines a facing surface 138 configured to face and/or contact the tissue wall when fixation mechanism 114 attaches to the tissue wall, and the curvature C is a positive curvature with respect to facing surface 138.

In examples, electrode 116 is a contact electrode configured to contact heart surface 124 in a relatively non-penetrating manner. Leadlet 108 may be configured such that at least some portion of leadlet body 122 and/or electrode 116 substantially lays atop heart surface 124 when fixation mechanism 114 attaches to the tissue of heart 102. The resilient biasing of shape memory material 130 may tend to urge leadlet body 122 toward heart surface 124 when the portion of leadlet body 122 and/or electrode 116 substantially lays atop heart surface 124, such that leadlet body 122 substantially maintains electrode 116 in contact with heart surface 124. In examples, leadlet 108 mechanically supports electrode 116 in a distal portion of leadlet body 122 that defines leadlet distal end 120. In examples, leadlet 108 mechanically supports electrode 116 substantially at leadlet distal end 120. In some examples, leadlet 108 mechanically supports electrode 116 such that electrode 116 substantially defines leadlet distal end 120. Leadlet body 122 may be configured such that, when fixation mechanism 114 attaches to tissue, the preset orientation of leadlet body 122 causes leadlet body 122 to position electrode 116 in contact with heart surface 124.

Circuitry 126 (shown in dashed lines) may be configured to deliver therapy signals and/or sense signals using electrode 116. In examples, circuitry 126 is mechanically supported by device body 106 (e.g., in a hermetically sealed enclosure defined by device body 106), although this is not required. Leadlet body 122 and/or device body 106 may mechanically support one or more conductors such as conductor 132 (shown in dashed lines) in electrical communication with circuitry 126 and electrode 116. Conductor 132 may be configured such that circuitry 126 may electrically communicate with (e.g., deliver therapy signals to and/or sense signal from) electrode 116. In examples, as will be discussed, leadlet body 122 and/or device body 106 mechanically supports a plurality of electrodes and/or a plurality of conductors, with each conductor electrically connected to at least one electrode.

In examples, device body 106 includes a housing 134 extending along longitudinal axis L substantially from device proximal end 105 to device distal end 107. Housing 134 may be formed from a biocompatible and biostable metal such as titanium. In some examples, housing 134 includes a hermetically sealed housing. In some examples, housing 134 includes a nonconductive coating and defines return electrode 128 as an uncoated portion of housing 134. Device body 106 may include a distal cap 136 configured to be secured to housing 134 during, for example, assembly of IMD 104. Distal cap 136 may be configured such that distal cap 136 and housing 134 act as a substantially unified body when distal cap 136 is secured to housing 134. Distal cap 136 may be configured to secure to housing 134 by welding, soldering, an adhesive, mechanical mating, or some other method.

Housing 134 may surround and/or define a hermetically sealed enclosure mechanically supporting circuitry 126. In some examples, rather than or in addition to circuitry 126 mechanically supported by device body 106, IMD 104 may include an implantable or external lead (not shown) configured to extend from device body 106 to an external device located outside of heart 102 (FIG. 1), with the external device including circuitry configured to deliver therapy signals to and/or sense signals from electrode 116 using the implantable or external lead.

Fixation mechanism 114 is configured to engage tissue at a target site (e.g., target site 112 (FIG. 1)) to secure IMD 104 to the tissue. Fixation mechanism 114 is configured such that, when fixation mechanism 114 engages tissues and a force in the proximal direction P is exerted on device body 106, fixation mechanism 114 exerts a reaction force in the distal direction D on device body 106, tending to limit movement of device body 106. Fixation mechanism 114 may be configured to substantially secure device body 106 (e.g., device distal end 107) in a position relative to the tissues at the target site, such that the preset orientation of leadlet body 122 causes electrode 116 to contact heart surface 124 when fixation mechanism 114 secures to the tissue. Fixation mechanism 114 may include, for example, one or more elongated tines such as fixation tine 113 and/or fixation tine 115 configured to substantially engage the tissue at a target site. Fixation mechanism 114 may include fixation tines of any shape, including helically-shaped fixation tines. In examples, fixation mechanism 114 is configured to substantially maintain contact between electrode 116 and tissues within a target site when fixation mechanism 114 engages the tissue. Fixation mechanism 114 may be configured to position within the cup section of delivery catheter 110 (FIG. 1) when IMD 104 is positioned within the cup section, with one or more tines such as tine 113, 115 resiliently biased to deploy outward to grasp tissue when delivery catheter 110 is proximally withdrawn (e.g., by a clinician).

In examples, IMD 114 includes a distal electrode 121 extending from a distal portion (e.g., distal cap 136) of device body 136. Distal electrode 121 may be configured to flexibly maintain contact with the wall tissue of heart 102 at or near target site 112 substantially without penetration of the wall tissue by distal electrode 121. Distal electrode 121 may be configured to flexibly maintain contact with the wall tissue despite variations in the tissue surface or in the distance between device distal end 107 and the tissue surface, which may occur as the wall tissue moves during the cardiac cycle. In order to flexibly maintain contact with the wall tissue, distal electrode 121 may be flexible and configured to have spring-like properties. For example, distal electrode 121 may be configured to elastically deform, e.g., toward device distal end 107, but may be spring biased toward a resting configuration and, when elastically deformed, the spring bias may urge the second electrode away from device distal end 107. In this manner, the elastic deformation and spring bias may maintain distal electrode 121 in consistent contact with the wall tissue.

In examples, for example when IMD 104 includes distal electrode 121, fixation element 114 is a helically shaped fixation element. In some examples, distal electrode 121 is configured as a partial helix, e.g., a helix that does not make a full revolution around a longitudinal axis L. In examples, distal electrode 121 includes one or more electrically insulating coatings, e.g., a parylene, polyurethane, silicone, epoxy, or other insulating coating, to reduce an electrically conductive active surface area of distal electrode 121 and define an electrically active area of distal electrode 121. As described herein, to flexibly maintain contact generally refers to an electrode being moveable with respect to housing 134. For example, an electrode may be configured to elastically deform as described above. In some examples, an electrode may additionally be attached to housing 134 by, or may include, a mechanism, such as a spring or joint, that allows relative motion of the electrode to housing 134. In such examples, the electrode need not itself be deformable.

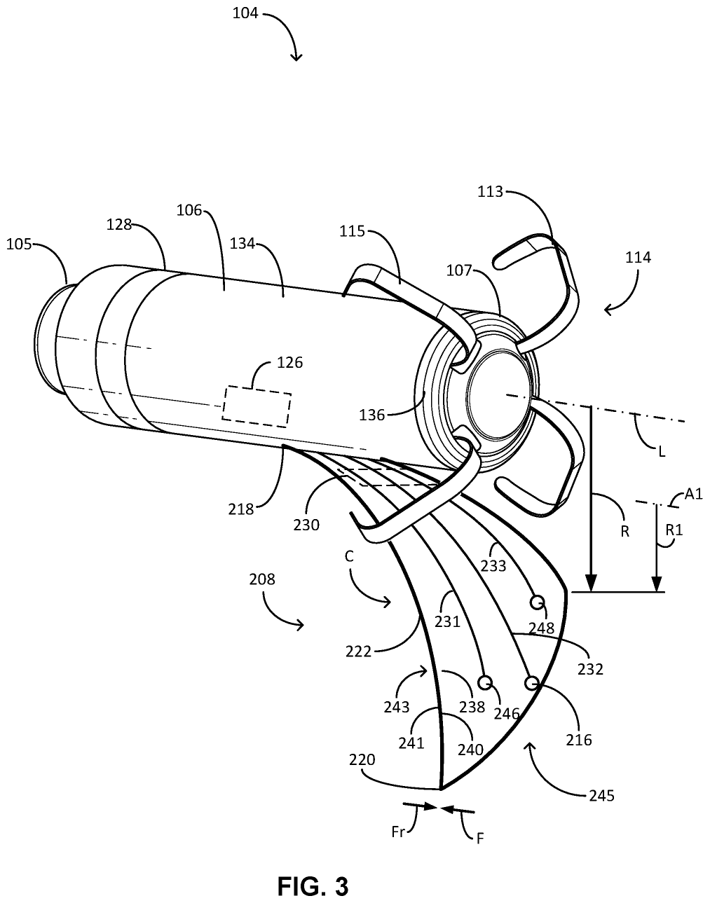

FIG. 3 illustrates a leadlet 208 of IMD 104 mechanically coupled to device body 106 at a location substantially between device distal end 107 and device proximal end 105. Leadlet 208 includes a leadlet body 222 mechanically supporting an electrode 216 electrically connected to circuitry 126 by a conductor 232. Leadlet body 222 includes a proximal end 218 (“leadlet proximal end 218”) mechanically coupled to device body 106 and a distal end 220 (“leadlet distal end 220”) opposite leadlet proximal end 218. Leadlet 208 includes leadlet body 222, leadlet proximal end 218, leadlet distal end 220, shape memory material 230, and facing surface 238, and mechanically supports electrode 216 and conductor 232. Leadlet 208, leadlet body 222, leadlet proximal end 218, leadlet distal end 220, electrode 216, conductor 232, shape memory material 230, and facing surface 238 may be examples of leadlet 108, leadlet body 122, leadlet proximal end 118, leadlet distal end 120, electrode 116, conductor 132, shape memory material 130, and facing surface 138. FIG. 3 illustrates leadlet body 222 in a relaxed configuration, such that leadlet body 222 is substantially free of forces external to IMD 104 acting on leadlet body 222, and any stresses on or within leadlet body 222 arise from properties or phenomena internal to leadlet body 222.

Leadlet body 222 is configured such that shape memory material 230 causes leadlet body 222 to substantially establish a preset orientation relative to device body 106. In examples, leadlet body 222 is configured such that shape memory material 230 urges leadlet body 222 toward the preset orientation when leadlet body 222 departs from the preset orientation. Leadlet 208 is configured such that the preset orientation causes leadlet body 222 to define the radial displacement R between leadlet distal end 220 and longitudinal axis L. In examples, leadlet 208 is configured such that the preset orientation causes leadlet body 222 to define the radial displacement R1 between leadlet distal end 220 and axis A1. Leadlet body 222 may be configured to cause facing surface 238 to face and/or contact a tissue wall when fixation mechanism 114 attaches to the tissue wall. In examples, leadlet body 222 is configured to define the curvature C to cause facing surface 238 to face and/or contact the tissue wall. Leadlet body 222 may be configured to define the curvature C as a positive curvature with respect to facing surface 238.

In examples, leadlet body 222 substantially defines a sheet extending from device body 106. Shape memory material 230 may be configured to cause the sheet to define the preset orientation relative to device body 106. In examples, leadlet body 222 defines a first side 240 and a second side 241 opposite first side 240. First side 240 may define facing surface 238. In examples, first side 240 defines facing surface 238 as a substantially planar surface. Second side 241 may define a second surface 243. In examples leadlet body 222 is configured to position facing surface 238 facing substantially toward heart surface 124 (FIG. 1) and position second surface 244 facing substantially away from heart surface 124 when fixation mechanism 114 attaches to tissues of heart 102. Leadlet body 222 may be configured to cause facing surface 238 to face substantially toward heart surface 124 and position second surface 244 to face substantially away from heart surface 124 when leadlet body 222 substantially establishes the preset orientation and/or is urged toward the preset orientation. In examples, electrode 216 is configured to contact heart surface 124 when facing surface 238 contacts heart surface 124.

Leadlet body 222 may mechanically support a plurality of electrodes 245. For example, leadlet body 222 may mechanically support electrode 246 and electrode 248 in addition to or instead of electrode 216. Leadlet body 222 may mechanically support electrodes 216, 246, 248 such that electrodes 216, 246, 248 define a distributed pattern across facing surface 238. In examples, leadlet body 222 is configured to cause two or more of electrodes 216, 246, 248 to contact heart surface 124 when fixation mechanism 114 secures to tissues of heart 102, such that electrodes 216, 246, 248 define a distributed pattern in contact with heart surface 124 when fixation mechanism 114 secures to tissues of heart 102 (e.g., secures to tissues within or in the vicinity of target site 112 (FIG. 1)).

IMD 104 may be configured to utilize electrode 216, 246, 248 to evaluate and/or enhance therapy delivered to heart 102 from circuitry 126. In examples, IMD 104 is configured to individually communicate with (e.g., deliver individual signals to and/or sense individual signals from) each of electrodes 216, 246, 248 to conduct, for example, pace mapping of heart 102. For example, IMD 104 may be configured to individually communicate with each of electrodes 216, 246, 248 when fixation mechanism 114 secures to heart 102 in the vicinity of target site 112 (FIG. 1) and leadlet body 222 positions electrodes 216, 246, 248 in contact with heart surface 124. The individual communication may allow a clinician to, for example, evaluate a cardiac response to a signal emitted by one or more of electrodes 216, 246, 248 with fixation mechanism 114 secured in the vicinity of target site 112. The clinician may cause IMD 104 to utilize one or more of electrodes 216, 246, 248, such that the clinician may select electrodes delivering effective therapy to the patient when fixation mechanism 114 is secured in the vicinity of target site 112.

Leadlet body 222 may mechanically support a plurality of conductors, with each conductor electrically connected to at least one of electrodes 216, 246, 248. For example, in addition to or instead of conductor 232, leadlet body 222 may mechanically support a conductor 231 and a conductor 233. Conductor 231 may be electrically connected to electrode 246. Conductor 233 may be electrically connected to electrode 248. Each of conductors 231, 232, 233 may be electrically connected to circuitry 126, such that circuitry 126 may deliver therapy signals to and/or sense signals from any one or a combination of electrodes 216, 246, 248. In examples, leadlet body 222 is configured to electrically insulate an individual conductor of conductors 231, 232, 233 from any other conductor of conductors 231, 232, 233, such that circuitry 126 may deliver and/or sense a first signal using a first electrode and a first conductor (e.g., electrode 216 and conductor 232) and deliver and/or sense a second signal using a second electrode and a second conductor (e.g., electrode 246 and conductor 231). Circuitry 126 may be configured to utilize one or more of conductors 231, 232, 233 based on a received instruction (e.g., from a clinician), such that IMD 104 may utilize one of or a specific combination of electrodes 216, 246, 248 to deliver therapy to the patient.

In examples, leadlet body 122, 222 is configured to substantially extend leadlet distal end 120, 220 beyond a radial displacement defined by device body 106. Leadlet body 122, 222 may be configured to position electrode 116, 216, 246, 248 such that electrical contact between electrode 116, 216, 246, 248 and the tissues of the heart 102 occurs at a location displaced beyond the radial displacement defined by device body 106. The radial displacement of device 106 may be a displacement defined by a vector perpendicular to and extending from longitudinal axis L to a fixed point on device body 106. In examples, the radial displacement of device 106 is the maximum displacement defined by a vector perpendicular to and extending from longitudinal axis L to a fixed point on device body 106. In examples, the radial displacement is a cross-sectional dimension of device body 106 (e.g., a dimension of a cross-section substantially perpendicular to longitudinal axis L).

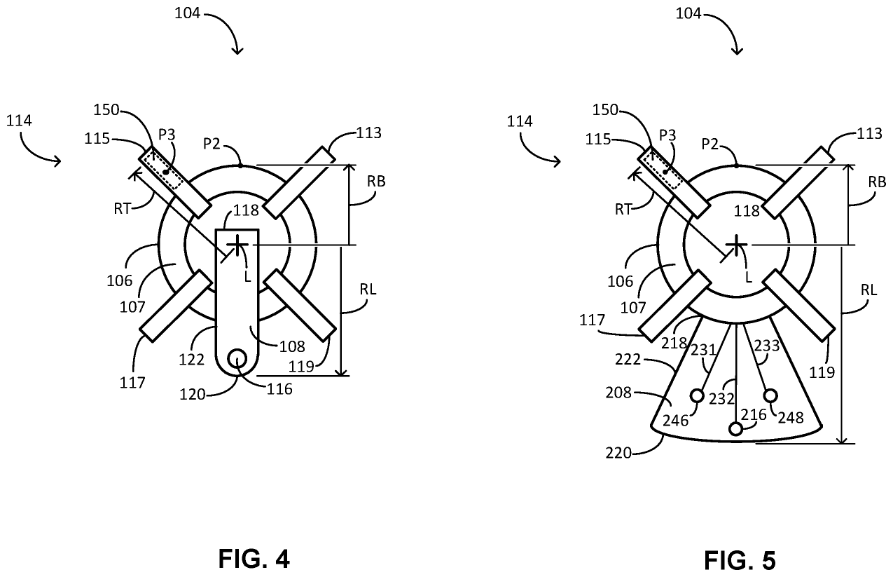

For example, FIG. 4 illustrates an end view of IMD 104, oriented such that longitudinal axis L is perpendicular to the page. Device body 106 defines a maximum radial displacement RB from longitudinal axis L to a fixed point P2 on device body 106. Point P2 is a point located on device body 106 which defines a maximum displacement of device body 106 from longitudinal axis L in a direction perpendicular to longitudinal axis L. In examples, the maximum radial displacement RB is a cross-sectional dimension of device body 106.

Leadlet 108 may be configured to substantially extend leadlet distal end 120 beyond a radial displacement defined by device body 106. In examples, leadlet 108 is configured to substantially extend leadlet distal end 120 beyond the maximum radial displacement RB. Leadlet 108 may be configured such that the preset orientation of leadlet 108 causes leadlet body 122 to define a radial displacement RL between leadlet distal end 120 and longitudinal axis L. Radial displacement RL may be substantially perpendicular to longitudinal axis L. In some examples, the radial displacement RL may be substantially equal to the radial displacement R (FIG. 2) defined when leadlet body 122 substantially establishes and/or is urged toward the preset orientation caused by shape memory material 130. In other examples, the radial displacement RL may be a radial displacement defined when an external force on leadlet body 122 (e.g., the force F (FIG. 2) causes leadlet body 122 to depart from the preset orientation (e.g., to substantially flatten, such that leadlet body 122 moves proximally toward device distal end 107). The radial displacement RL defined by leadlet body 122 may be greater than a radial displacement and/or the maximum radial displacement RB defined by device body 106, such that leadlet 108 substantially extends leadlet distal end 120 beyond the maximum radial displacement RB. In examples, leadlet body 122 is configured to position electrode 116 such that electrical contact between electrode 116 and the tissues of the heart 102 occurs at a location displaced beyond the radial displacement RB. In examples, fixation mechanism includes a first tine such as fixation tine 117 and a second tine such as fixation tine 119, and leadlet 108 is configured to pass between the first tine and the second tine when leadlet body 122 defines the radial displacement RL. Fixation tine 117, 119 may be examples of and/or configured similarly to fixation tine 113, 115.

In examples, the radial displacement RL defined by leadlet body 122 is greater than a radial displacement between the longitudinal axis L and an attachment site of fixation mechanism 114 when fixation mechanism 114 secures IMD 104 to a tissue wall. The attachment site may be a site on a tissue surface where fixation mechanism 114 penetrates the tissue wall. In examples, fixation mechanism 114 defines an attachment area on fixation mechanism 114, with the attachment area defining a location on fixation mechanism 114 where fixation mechanism 114 passes through the tissue surface when fixation mechanism 114 secures IMD 104 to the tissue wall. Stated similarly, fixation mechanism 114 may be configured such that, when fixation mechanism 114 penetrates the tissue surface and inserts into the tissue wall, the attachment area defines a non-penetrating portion of fixation mechanism 114 between the attachment area and device body 106, with the non-penetrating portion configured to remain outside the tissue wall when fixation mechanism 114 secures IMD 104 to the tissue wall.

For example, FIG. 4 illustrates an attachment area 150 defined on fixation tine 115 of fixation mechanism 114. Attachment area 150 includes a point on fixation tine 115 where fixation tine 115 passes through the tissue surface when fixation mechanism 114 secures IMD 104 to the tissue wall. For example, attachment area 150 may include a point P3 on tine 115 wherein fixation tine 115 is configured to pass through the tissue surface when fixation mechanism 114 secures IMD 104 to the tissue wall. Point P3 may be anywhere within attachment area 150. Hence, attachment area 150 may define a radial displacement RT, wherein the radial displacement RT defines a maximum radial displacement expected between longitudinal axis L and a non-penetrating portion of fixation tine 115 when fixation tine 115 penetrates the tissue wall. In examples, the radial displacement RL defined by leadlet body 122 is greater than the radial displacement RT defined by fixation mechanism 114, such that leadlet 108 may substantially extend leadlet distal end 120 beyond the attachment site of fixation mechanism 114 when fixation mechanism 114 secures IMD 104 to a tissue wall. In examples, leadlet body 122 is configured to position electrode 116 such that electrical contact between electrode 116 and the tissues of the heart 102 occurs at a location displaced beyond the radial displacement RT.

FIG. 5 illustrates an end view of IMD 104 including leadlet 208 and oriented such that longitudinal axis L is perpendicular to the page. Leadlet 208 may be configured such that the preset orientation of leadlet 208 causes leadlet body 222 to define the radial displacement RL between leadlet distal end 220 and longitudinal axis L. The radial displacement RL may be substantially equal to the radial displacement R (FIG. 3) defined when leadlet body 222 substantially establishes and/or is urged toward the preset orientation caused by shape memory material 230. In other examples, the radial displacement RL may be a radial displacement defined when an external force on leadlet body 222 (e.g., the force F (FIG. 3) causes leadlet body 222 to depart from the preset orientation. The radial displacement RL defined by leadlet body 222 may be greater than the radial displacement and/or the maximum radial displacement RB defined by device body 106, such that leadlet 208 substantially extends leadlet distal end 220 beyond the maximum radial displacement RB. In examples, leadlet body 222 is configured to position one or more of electrode 216, 246, 248 such that electrical contact between electrodes 216, 246, 248 and the tissues of the heart 102 occurs at a location displaced beyond the radial displacement RB.

In examples, the radial displacement RL defined by leadlet body 222 is greater than the radial displacement between the longitudinal axis L and the attachment site of fixation mechanism 114 (e.g., fixation tine 113) when fixation mechanism 114 secures IMD 104 to a tissue wall. In examples, the radial displacement RL defined by leadlet body 222 is greater than the radial displacement RT defined by fixation mechanism 114, such that leadlet 208 may substantially extend leadlet distal end 220 beyond the attachment site of fixation mechanism 114 when fixation mechanism 114 secures IMD 104 to a tissue wall. In examples, leadlet body 222 is configured to position one or more of electrode 216, 246, 248 such that electrical contact between electrode 216, 246, 248 and the tissues of the heart 102 occurs at a location displaced beyond the radial displacement RT.

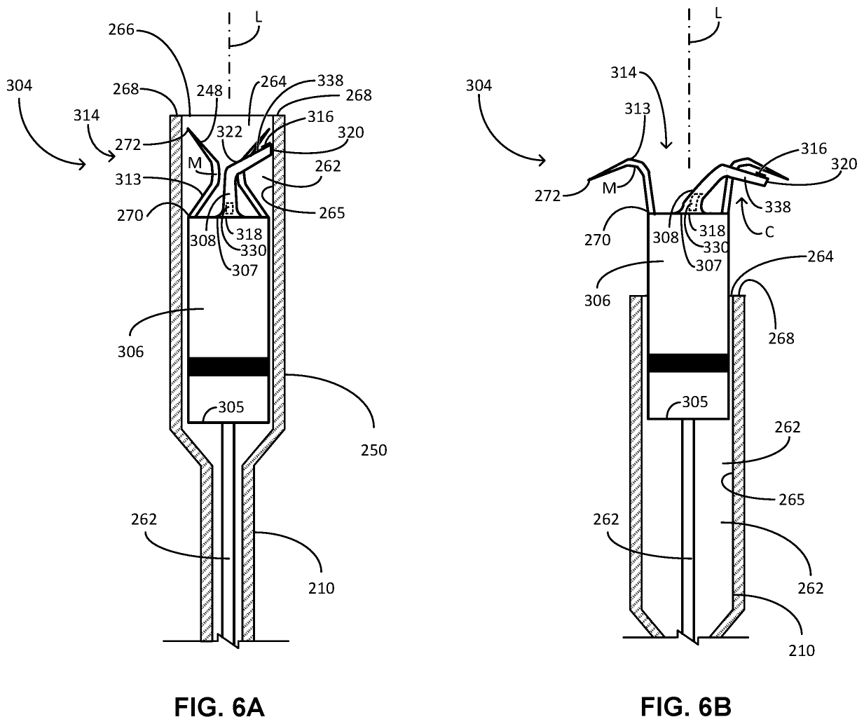

As discussed, IMD 104 may be configured to position within a delivery catheter for delivery to a target site (e.g., target site 112) within a patient. In examples, IMD 104 may be configured to position within a cup section in a distal portion of the delivery catheter. As an example, FIG. 6A illustrates an example IMD 304 positioned within a cup section 262 of a delivery catheter 210. FIG. 6B illustrates IMD 304 with delivery catheter 210 and cup section 262 proximally displaced from IMD 304. Delivery catheter 210 is illustrated as a cross-section with a cutting plane parallel to the page. IMD 304 includes a leadlet 308. IMD 304 is an example of IMD 104. Leadlet 308 is an example of leadlet 108, 208. Delivery catheter 210 is an example of delivery catheter 110. IMD 304 further includes device body 306, device proximal end 305, device distal end 307, leadlet proximal end 318, leadlet distal end 320, leadlet body 322, shape memory material 330, facing surface 338, electrode 316, and fixation mechanism 314 with fixation tine 313, which may be configured individually and in relation to each other in the same manner as that described for like-named components of IMD 104.

Cup section 262 may define a lumen 264 configured to at least circumferentially surround IMD 304, such that delivery catheter 210 may deliver IMD 304 to heart 102 (FIG. 1). In examples, cup section 262 includes an inner wall 265 defining lumen 264. Cup section 262 may define a lumen opening 266 opening to lumen 264 at a distal end 268 of cup section 262 (“cup distal end 268”) configured such that fixation mechanism 314, device body 306, and leadlet 308 may pass therethrough. Leadlet 308 may be configured to substantially establish and/or be urged toward the preset orientation as leadlet 308 passes through lumen opening 266. For example, IMD 304 may be configured such that a portion of leadlet body 322 (e.g., leadlet distal end 320) contacts inner wall 265 when lumen 264 circumferentially surrounds IMD 304. Inner wall 265 may exert a force on leadlet body 322 causing leadlet body 322 to depart from the preset orientation when lumen 264 circumferentially surrounds IMD 304. Shape memory material 330 may be resiliently biased to cause leadlet body 322 to expand outward as leadlet body 322 passes through lumen opening 266, to cause leadlet body 322 to substantially establish the preset orientation and/or urged leadlet body 322 toward the preset orientation. Cup section 262 may be configured to radially constrain leadlet body 322 when IMD 304 (e.g., leadlet body 322) is proximal to lumen opening 266.

In examples, shape memory material 330 is configured to drive at least leadlet distal end 320 radially outward from longitudinal axis L as leadlet body 322 passes through lumen opening 266, as illustrated in FIG. 6B. The resilient biasing of shape memory material 330 tending to cause leadlet distal end 320 to radially displace outward as leadlet body 322 extends through lumen opening 266 may cause leadlet body 322 to substantially establish the preset orientation and cause facing surface 338 and/or electrode 316 to substantially face toward a tissue wall located distal to lumen opening 266. In examples, resilient biasing of shape memory material 330 may urge leadlet body 322 toward the preset orientation and cause facing surface 338 and/or electrode 316 to substantially face toward a tissue wall located distal to lumen opening 266. The resilient biasing of shape memory material 330 may cause leadlet body 322 to define the curvature C between leadlet proximal end 318 and leadlet distal end 320. As previously discussed, leadlet proximal end 318 may be attached to any location on device body 306. Leadlet proximal end 318 may be attached to device distal end 307, device proximal end 305, or to a portion of device body 306 between device distal end 307 and device proximal end 305.

Fixation mechanism 314 may be configured to engage tissue (e.g., within target site 112 (FIG. 1)) as fixation mechanism 314 passes through lumen opening 266. In examples, fixation mechanism 314 (e.g., fixation tine 313) is configured to extend distally from device body 306 when IMD 304 is positioned within cup section 262. Fixation mechanism 314 may be configured to penetrate tissues as fixation mechanism 314 passes through lumen opening 266 in order to engage the tissues. For example, a portion of fixation mechanism 314 (e.g., fixation tine 313) may be resiliently biased to expand outward as fixation mechanism 314 passes through lumen opening 266, in order to aid in grasping the tissue. Cup section 262 may be configured to radially constrain fixation mechanism 314 (e.g., fixation tine 313) when fixation mechanism 314 is proximal to lumen opening 266. Fixation mechanism 314 may be configured to position within cup section 262 when IMD 304 is positioned within the cup section, with one or more tines such as tine 313 resiliently biased to deploy outward to grasp tissue when delivery catheter 210 is proximally withdrawn (e.g., by the clinician).

In examples, fixation tine 313 includes a fixed end 270 mechanically supported by device body 306 and a free end 272 opposite fixed end 270. Free end 272 may be configured to penetrate tissue. In examples, fixation tine 313 is biased to drive free end 272 radially outward from longitudinal axis L of IMD 304 as free end 272 passes through lumen opening 266, as illustrated in FIG. 6B. The biasing tending to drive free end 272 radially outward as fixation tine 313 extends through lumen opening 266 may cause fixation tine 313 to substantially grasp tissue (e.g., within heart 102). Free end 272 may pierce the tissue and may act to pull IMD 304 toward a target site as fixation tine 313 elastically bends or curves radially outward. Fixation mechanism 314 may include any number of fixation tines, which may be configured similarly to fixation tine 313.

The biasing of fixation tine 313 tending to drive free end 272 radially outward may cause fixation tine 313 to assume any general shape. In some examples, the biasing of fixation tine 313 tends to cause fixation tine 313 to position such that free end 272 establishes a position distal to a midpoint M between fixed end 270 and free end 272 (e.g., as depicted in FIG. 6B). In some examples, the biasing of fixation tine 313 tends to cause fixation tine 313 to position such that free end 272 establishes a position proximal to midpoint M. Fixation tine 313 may be formed to have a preset shape and may be formed using any suitable material. In examples, fixation tine 313 comprises a nickel-titanium alloy such as Nitinol.

In some examples, fixation tine 313 may be configured to substantially maintain a delivery configuration where free end 272 is distal to fixed end 270 and distal to midpoint M (e.g., as depicted in FIG. 6A). For example, fixation tine 313 may be configured to substantially maintain the delivery configuration when free end 272 is constrained from outward radial motion by inner wall 265. Cup section 250 may be configured to substantially maintain fixation tine 313 in the delivery configuration as delivery catheter 210 translates through vasculature to deliver IMD 304 to heart 102. Substantially maintaining free end 272 distal to midpoint M (e.g., in the delivery configuration) may facilitate the penetration of tissue by free end 272 when fixation tine 313 passes through lumen opening 266 of delivery catheter 18. Fixation tine 313 may refer to any structure that is capable of securing a lead or leadless implantable medical device to a location within the heart. In some examples, a tine (e.g., fixation tine 313) may be composed of a shape-memory allow that allows deformation along the length of the tine. A tine may be substantially flat along the length of the tine. In other examples, a tine may substantially define a helix and/or helical member.

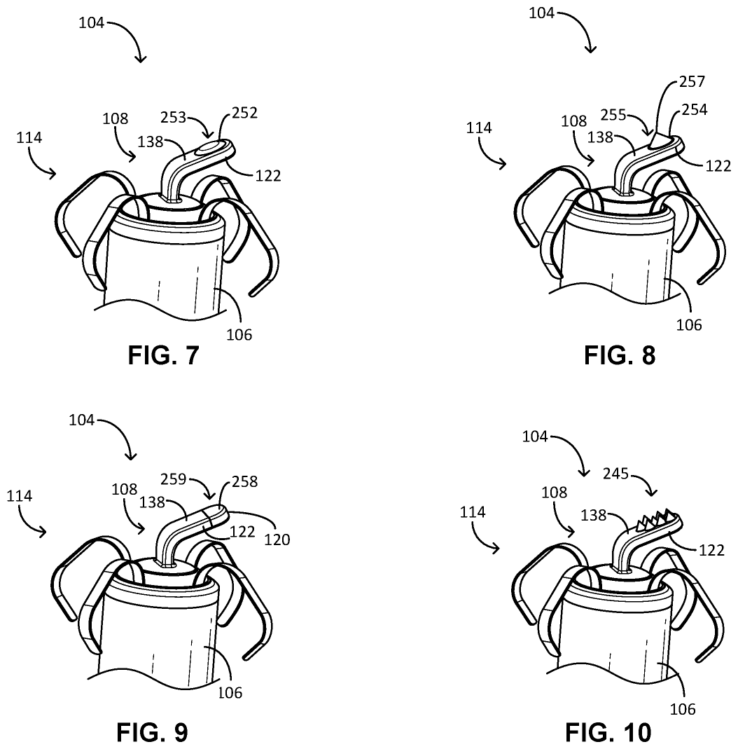

The electrode (e.g., electrode 116, 216, 246, 248) mechanically supported by leadlet 108, 208 may be configured in any manner to establish electrical communication with tissues of a heart (e.g., heart surface 124 (FIG. 1)) when the electrode contacts the tissues of the heart. For example, FIG. 7 illustrates an electrode 252 mechanically supported by leadlet 108 and defining a contact area 253. Electrode 252 is configured to electrically communicate with heart surface 124 when contact area 253 contacts heart surface 124. Electrode 252 may be configured such that contact area 253 is a substantially smooth surface, such that electrode 252 maintains slidable contact with heart surface 124 when heart surface 124 moves relative to leadlet 108, 208 and/or electrode 252.

In some examples, the electrode may be configured to defines an apex configured to maintain electrical communication with heart surface 124. For example, FIG. 8 illustrates an electrode 254 wherein contact area 255 defines an apex 257. Apex 257 may be configured to substantially protrude from contact area 255, In examples, electrode 254 is configured such that apex 257 protrudes toward heart surface 124 when the preset orientation of leadlet 108, 208 places electrode 254 in contact with heart surface 124. In examples, electrode 254 is configured such that apex 257 defines a point on contact area 253 defining a maximum altitude between contact area 253 and a surface of leadlet body 122, 222 (e.g., facing surface 138, 238), where the altitude is measured over a line perpendicular to the surface.

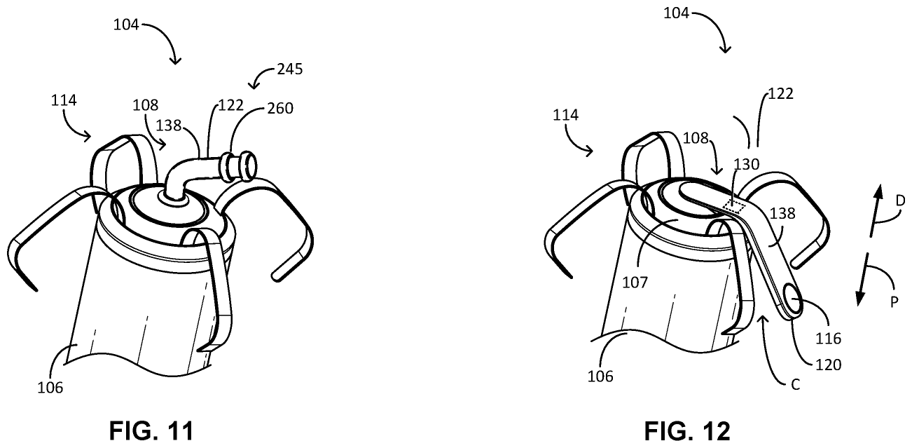

The electrode may include a contact area substantially defining leadlet distal end 120, 220. The contact area may substantially surround at some portion of a perimeter defined by leadlet 108, 208. For example, FIG. 9 illustrates an electrode 258 including contact area 259, where contact area 259 substantially extends over and defines leadlet distal end 120. Contact area 259 is configured to substantially surround at least a portion of a perimeter defined by leadlet 108 (e.g., a perimeter extending around facing surface 138 and a surface of leadlet 108 opposite facing surface 138). In some examples, as illustrated in FIG. 10, leadlet 108 may mechanically support plurality of electrodes 245. In some examples, as illustrates in FIG. 11, the plurality of electrode 245 may include one or more ring electrodes such as electrode 260 configured to surround the perimeter defined by leadlet 108. In examples, leadlet body 122 defines a substantially ovalur (e.g., oval-shaped or circular) perimeter, and electrode 260 is configured to surround the ovalur perimeter. Electrode 252, 254, 258, 260 may be an example of any of electrode 116, 216, 246, 248. Electrode 252, 254, 258, 260, 116, 216, 246, 248 may have any surface texture. For example, electrode 252, 254, 258, 260, 116, 216, 246, 248 may include one or more electrical conductor surfaces (e.g., active surfaces) having a substantially “rough” or uneven surface.

Leadlet 108, 208 may be configured to displace leadlet distal end 120, 220 distal or proximal to device body 106 when leadlet 108, 208 substantially establishes and/or is urged toward the preset orientation. In examples, leadlet 108, 208 is configured to displace leadlet distal end 120, 220 and/or electrode 216, 242, 246, 248, 252, 254, 258, 260 substantially distal or proximal to device distal end 107 when leadlet 108, 208 substantially establishes and/or is urged toward the preset orientation. For example, FIG. 2 illustrates leadlet 108 displacing leadlet distal end 120 and/or electrode 116 distal to (e.g., in the distal direction D) device distal end 107 when leadlet 108 substantially establishes and/or is urged toward the preset orientation. Leadlet 108 may be configured to displace leadlet distal end 120 and/or electrode 116 proximal to device distal end 107 when leadlet 108 defines the curvature C. In contrast, FIG. 12 illustrates leadlet 108 displacing leadlet distal end 120 and/or electrode 116 proximal to (e.g., in the proximal direction P) device distal end 107 when leadlet 108 substantially establishes and/or is urged toward the preset orientation. In an example, when shape memory material 130 causes leadlet 108 to define the curvature C, leadlet 108 displaces leadlet distal end 120 and/or electrode 116 proximal to device distal end 107. Leadlet 108, 208 may be configured to displace leadlet distal end 120, 220 and/or electrode 216, 242, 246, 248, 252, 254, 258, 260 in any position relative to device distal end 107, including substantially distal to, substantially proximal to, or substantially even with device distal end 107 when leadlet 108, 208 substantially establishes and/or is urged toward the preset orientation.

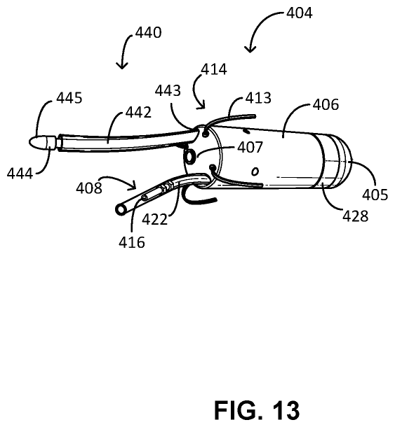

FIG. 13 illustrates an IMD 404 with additional leads and/or electrodes configured to deliver therapy to and/or sense signals from heart 102. The additional leads and/or electrodes may be configured as contact electrodes configured to contact a surface of heart 102 and/or may be configured as penetrating electrodes configured to penetrate and implant in tissues of heart 102. IMD 404 is an example of IMD 104, 304. IMD 404 further includes device body 406 mechanically supporting leadlet 408, device proximal end 405, device distal end 407, leadlet body 422, electrode 416, return electrode 428, and fixation mechanism 414 with fixation tine 413, which may be configured individually and in relation to each other in the same manner as that described for like-named components of IMD 104, 304.

IMD 404 includes a second leadlet 440 with a leadlet body 442 (“second leadlet body 442”) mechanically supporting an electrode 444. Electrode 444 may be operably coupled to circuitry 126 (FIGS. 1, 2, 3) via a conductor of second leadlet 440 (not shown). In some examples, electrode 444 is a penetrating electrode configured to penetrate and implant in tissues of heart 102 to electrically communicate with the tissues of heart 102. In some examples, electrode 444 is a contact electrode configured to contact a surface of heart 102 to electrically communicate with tissues of heart 102.

Second leadlet body 442 may define a proximal end 443 (“second proximal end 443”) and a distal end 445 (“second distal end 445”) opposite second proximal end 443. Electrode 444 may be mechanically supported on any portion of second leadlet body 442. In examples, electrode 444 substantially defines second distal end 445. Second proximal end 443 may be secured to device body 406 in any location, including a distal portion defining device distal end 407, a proximal portion defining device proximal end 405, or a portion of device body 406 substantially between device distal end 407 and device proximal end 405. In examples, second proximal end 443 is secured to device distal end 407. In examples, IMD 404 may include an implantable or external lead configured to extend from device body 306 to an external device located outside of heart 102. The external device may include circuitry 126 (FIGS. 1, 2, 3) configured to deliver therapy signals to and/or sense signals from electrodes 416, 444 using the implantable or external lead.

As discussed, circuitry 126 may be configured to deliver therapy to and/or sense cardiac signals from heart 102 (FIG. 1) using electrode 116, 216, 242, 246, 248, 252, 254, 258, 260, 316, 416, 444, and/or return electrode 128, 428. Circuitry 126 may be operably coupled to electrode 116, 216, 242, 246, 248, 252, 254, 258, 260, 316, 416, 444, and/or return electrode 128, 428 via one or more conductors. Circuitry 126 may be configured to transmit therapy signals using electrode 116, 216, 242, 246, 248, 252, 254, 258, 260, 316, 416, 444, and/or return electrode 128, 428, and may be configured to receive data representative of heart 102 from electrode 116, 216, 242, 246, 248, 252, 254, 258, 260, 316, 416, 444, and/or return electrode 128, 428. In examples, circuitry 126 includes one or more processors that are configured to implement functionality and/or process instructions stored in a storage device. Circuitry 126 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Circuitry 126 may include any suitable structure, whether in hardware, software, firmware, or any combination thereof, to perform the functions ascribed herein to the circuitry.

In examples, circuitry 126 is located within a housing of IMD 104, 304, 404. In other examples, circuitry 126 is located within another device or group of devices external to IMD 104, 304, 404 (e.g., within a device or group of devices not illustrated in FIGS. 1-13). As such, techniques and capabilities attributed herein to circuitry 126 may be attributed to any combination of IMD 104, 304, 404 and other devices that are not illustrated in FIGS. 1-13. Hence, medical system 100 (FIG. 1) may represent a system wherein portions are configured to be implanted within a patient and/or configured to be extracorporeal to a patient, and may include any fixed or mobile computer system (e.g., a controller, a microcontroller, a personal computer, minicomputer, tablet computer, etc.), and may be generally described as including substantially all or some portion of circuitry 126. For example, an implantable or external lead may be configured to connect to another IMD implanted in the patient at a location different than IMD 104, 304, 404, or to connect to a portion of medical system 100 extracorporeal to the patient.

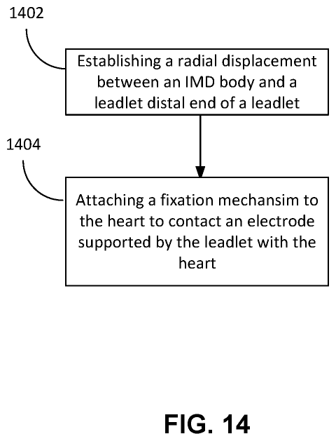

A technique for implanting an IMD 104, 204, 304 within a heart 102 is illustrated in FIG. 14. Although the technique is described mainly with reference to IMD 104, 204, 304, FIGS. 1-13, the technique may be applied to other medical devices in other examples.

The technique includes establishing a radial displacement between a leadlet distal end 120, 220, 320 of a leadlet 108, 208, 308, 408 and a device body 106, 306, 406 of IMD 104, 304, 404 using a shape memory material 130, 230, 330 (1402). Shape memory material 130, 230, 330 may cause leadlet 108, 208, 308, 408 to establish the radial displacement between leadlet distal end 120, 220, 320 and a longitudinal axis L extending through device body 106, 306, 406 of IMD 104, 304, 404. Longitudinal axis L may extend through device proximal end 105, 405 and device distal end 107, 407. In examples, leadlet 108, 208, 308, 408 defines a preset orientation with respect to device body 106, 306, 406 to cause the radial displacement between leadlet distal end 120, 220, 320 and device body 106, 306, 406. Shape memory material 130, 230, 330 may cause leadlet 108, 208, 308, 408 to define the preset orientation.

Shape memory material 130, 230, 330 may cause leadlet body 122, 222, 322, 422 to define the preset orientation. In examples, shape memory material 130, 230, 330 causes leadlet body 122, 222, 322, 422 to define the preset orientation when leadlet body 122, 222, 322, 422 is in a relaxed configuration. In examples, shape memory material 130 opposes an external force on leadlet body 122 causing leadlet body 122 to depart from the preset orientation. Shape memory material 130 may generate an internal stress and to urge leadlet body 122 to attempt to establish or reestablish the preset orientation when the external force causes a departure from the preset orientation. In examples, leadlet body 122, 222, 322, 422 substantially extends leadlet distal end 120, 220, 320 beyond a radial displacement defined by device body 106, 306, 406.

The technique includes attaching a fixation mechanism 114, 314, 414 to heart 102 and contacting electrode 116, 216, 242, 246, 248, 252, 254, 258, 260, 316, 416 mechanically supported by leadlet body 122, 222, 322, 422 to heart 102 (1404). Shape memory material 130, 230, 330 may urge leadlet body 122, 222, 322, 422 to establish the preset orientation to substantially maintain contact between electrode 116, 216, 242, 246, 248, 252, 254, 258, 260, 316, 416 and heart 102 (e.g., heart surface 124) when fixation mechanism 114, 314, 414 attaches to heart 102. In examples, leadlet body 122, 222, 322, 422 causes a radial displacement between electrode 116, 216, 242, 246, 248, 252, 254, 258, 260, 316, 416 and longitudinal axis L and/or axis A1 when leadlet 108 and/or leadlet body 122 substantially establishes and/or is urged toward the preset orientation. Leadlet body 122, 222, 322, 422 may cause the radial displacement between electrode 116, 216, 242, 246, 248, 252, 254, 258, 260, 316, 416 and longitudinal axis L and/or axis A1 when leadlet body 122, 222, 322, 422 defines the radial displacement R. Leadlet body 122, 222, 322, 422 may position electrode 116, 216, 242, 246, 248, 252, 254, 258, 260, 316, 416 to contact tissues of the heart 102 occurs at a location displaced beyond the radial displacement defined by device body 106, 306, 406.

Fixation tine 113, 313, 413 of fixation mechanism 114, 314, 414 may penetrate tissue of the heart 102 when fixation mechanism 114, 314, 414 attaches to heart 102. In examples, free end 272 penetrates tissue of heart 102. In examples, fixation tine 113, 313, 413 drives free end 272 radially outward from longitudinal axis L of IMD 304 as free end 272 passes through lumen opening 266 of cup section 262. Free end 272 may pierce the tissue and substantially pull IMD 104, 204, 304 toward target site 112 as fixation tine 113, 313, 413 elastically bends or curves radially outward. Leadlet 108, 208, 308, 408 may substantially establish and/or be urged toward the preset orientation as leadlet 108, 208, 308, 408 passes through lumen opening 266. In examples, fixation tine 113, 313, 413 expands radially outward from a delivery configuration caused by inner wall 265 of cup section 250 constraining an outward radial motion of fixation tine 113, 313, 413. In examples, leadlet body 122, 222, 322, 422 defines a radial displacement RL greater than a radial displacement between the longitudinal axis L and an attachment site of fixation mechanism 114, 314, 414 when fixation mechanism 114, 314, 414 secures IMD 104, 304, 404 to a tissue wall.

In examples, leadlet body 122, 222, 322, 422 substantially flattens (e.g., moves proximally) when fixation mechanism 114. 314. 414 attaches to target site 112 of heart 102. Leadlet body 122, 222, 322, 422 may be configured such that the proximal movement increases the radial displacement R. Leadlet body 122, 222, 322, 422 depart from the preset orientation when leadlet body 122, 222, 322, 422 substantially flattens. Shape memory material 130, 230, 330 may urge leadlet body 122 toward heart surface 124 to substantially maintain electrode 116, 216, 242, 246, 248, 252, 254, 258, 260, 316, 416 in contact with heart surface 124 when leadlet body 122, 222, 322, 422 departs from the preset orientation. In examples, leadlet body 122, 222, 322, 422 defines a curvature C between leadlet proximal end 118, 218, 318 and leadlet distal end 120, 220, 320 when leadlet body 122, 322, 422 substantially establishes and/or is urged toward the preset orientation.

Leadlet body 122, 222, 322, 422 may cause a plurality of electrode 116, 216, 242, 246, 248, 252, 254, 258, 260, 316, 416 to contact heart surface 124 when leadlet body 122, 222, 322, 422 substantially establishes and/or is urged toward the preset configuration. Leadlet body 122, 222, 322, 422 may cause electrode 116, 216, 242, 246, 248, 252, 254, 258, 260, 316, 416 to define a distributed pattern in contact with heart surface 124. In examples, leadlet body 122, 222, 322, 422 222 causes facing surface 138, 238 to face substantially toward heart surface 124 when leadlet body 122, 222, 322, 422 substantially establishes and/or is urged toward the preset configuration and fixation mechanism 114, 214, 314, 414 substantially attached to heart 102.

IMD 104 may evaluate and/or enhance therapy delivered to heart 102 from circuitry 126 using electrode 116, 216, 242, 246, 248, 252, 254, 258, 260, 316, 416. IMD 104 may individually communicate with each of electrode 116, 216, 242, 246, 248, 252, 254, 258, 260, 316, 416 using conductors 132, 232, 231, 233. In examples, circuitry 126 delivers and/or senses a first signal using a first electrode and a first conductor (e.g., electrode 216 and conductor 232) and delivers and/or senses a second signal using a second electrode a second conductor (e.g., electrode 246 and conductor 231). In examples, circuitry 126 receives an instruction (e.g., from a clinician) utilizes one of or a specific combination of electrodes 116, 216, 242, 246, 248, 252, 254, 258, 260, 316, 416 to deliver therapy to the patient.

The Disclosure Includes the Following Examples.

Example 1: A medical device comprising: a device body configured to position within a heart, the device body defining a device proximal end and a device distal end, and the device defining a longitudinal axis extending between the device proximal end and the device distal end; a fixation mechanism attached to a device distal end, wherein the fixation mechanism is configured to attach to tissue of the heart; and a leadlet mechanically supporting an electrode, wherein the leadlet defines a leadlet proximal end, a leadlet distal end, and a leadlet body between the leadlet proximal end and the leadlet distal end, wherein the leadlet proximal end is attached to the device body, wherein the leadlet body comprises a shape memory material configured to urge the leadlet body toward a preset orientation relative to the device body, and wherein the leadlet is configured to define a radial displacement between the leadlet distal end and the longitudinal axis when the shape memory material urges the leadlet body toward the preset orientation.

Example 2: The medical device of example 1, wherein the electrode is configured to contact a surface of the heart when the fixation mechanism attaches to the tissues of the heart and the shape memory material urges the leadlet body toward the preset orientation.

Example 3: The medical device of example 1 or example 2, wherein the shape memory material is configured to generate an internal stress tending to oppose an external force exerted on the leadlet body when the shape memory material urges the leadlet body toward the preset orientation.

Example 4: The medical device of example 3, wherein the internal stress acts on the shape memory material to cause the shape memory material to urge the leadlet body toward the preset orientation when the external force is exerted on the leadlet body.

Example 5: The medical device of any of examples 1-4, wherein the leadlet body defines a curvature between the leadlet proximal end and the leadlet distal end configured to cause the electrode to contact a surface of the heart when the fixation mechanism is attached to the tissues of the heart and the shape memory material urges the leadlet body toward the preset orientation.

Example 6: The medical device of any of examples 1-5, wherein the leadlet body defines a facing surface configured to substantially face a surface of the heart when the fixation mechanism is attached to the tissues of the heart and the shape memory material urges the leadlet body toward the preset orientation.

Example 7: The medical device of any of examples 1-6, wherein the device body defines a maximum radial displacement from the longitudinal axis, and wherein the radial displacement between the leadlet distal end and the longitudinal axis is greater than the maximum device radial displacement.

Example 8: The medical device of any of examples 1-7, wherein the preset orientation causes the leadlet body to extend toward the surface of the heart when the fixation mechanism is attached to the tissues of the heart.

Example 9: The medical device of any of examples 1-8, wherein the leadlet is configured to cause the electrode to contact a surface of the heart when the fixation mechanism is attached to the tissues of the heart and the shape memory material urges the leadlet body toward the preset orientation.

Example 10: The medical device of any of examples 1-9, wherein the device defines a distal direction from the device proximal end to the device distal end, and wherein the leadlet is configured to displace the leadlet distal end in a position distal to the leadlet proximal end when the shape memory material urges the leadlet body toward the preset orientation.

Example 11: The medical device of any of examples 1-9, wherein the device defines a proximal direction from the device distal end to the device proximal end, and wherein the leadlet is configured to displace the leadlet distal end in a position proximal to the leadlet proximal end when the shape memory material urges the leadlet body toward the preset orientation.

Example 12: The medical device of any of examples 1-11, wherein the leadlet is configured to radially displace the electrode from the longitudinal axis when the leadlet defines the radial displacement between the leadlet distal end and the longitudinal axis.

Example 13: The medical device of any of examples 1-12, wherein the leadlet proximal end is attached to the device distal end.

Example 14: The medical device of any of examples 1-12, wherein the device defines a proximal direction from the device distal end to the device proximal end, and wherein the leadlet proximal end is attached to a portion of the device body that is proximal to the device distal end.

Example 15: The medical device of any of examples 1-14, further comprising a conductor mechanically supported by the leadlet body, wherein the conductor is electrically connected to the electrode.

Example 16: The medical device of example 15, wherein the leadlet is configured to radially displace the conductor from the longitudinal axis when the leadlet defines the radial displacement between the leadlet distal end and the longitudinal axis.

Example 17: The medical device of example 15 or example 16, further comprising circuitry configured to deliver therapy signals to the heart using the electrode, wherein the conductor is electrically connected to the circuitry.

Example 18: The medical device of any of examples 15-17, wherein the leadlet body defines an insulative coating covering at least some portion of the conductor.

Example 19: The medical device of example 18, wherein the electrode is a portion of the conductor not covered by the insulative covering.

Example 20: The medical device of any of examples 1-19, wherein the shape memory material comprises a polymer.

Example 21: The medical device of any of examples 1-22, wherein the fixation mechanism includes one or more tines, wherein a tine includes a fixed end and a free end, wherein the fixed end is mechanically coupled to the device body, and wherein the tine is biased to drive the free end radially outward from the longitudinal axis.

Example 22: The medical device of example 21, wherein: the one or more tines includes a first tine and a second tine, the leadlet is secured to the device distal end, and the leadlet body is configured to pass between the first tine and the second tine when the leadlet body substantially establishes the preset orientation.

Example 23: The medical device of any of examples 1-22, wherein the electrode is a first contact electrode, and further comprising: a first conductor mechanically supported by the leadlet body, wherein the first conductor is electrically connected to the first contact electrode; a second contact electrode mechanically supported by the leadlet body; and a second conductor mechanically supported by the leadlet body, wherein the second conductor is electrically connected to the second contact electrode.

Example 24: The medical device of example 23, wherein: the first conductor is electrically connected to circuitry of the medical device, the second conductor is electrically connected to the circuitry of the medical device, and the circuitry is configured to deliver therapy signals to the heart using at least one of the first contact electrode or the second contact electrode.

Example 25: The medical device of any of examples 1-24, wherein the leadlet body is an elongated body defining a substantially circumference around the leadlet between the leadlet proximal end and the leadlet distal end, wherein the circumference is at least one of ovalur, polygonal, or a shape having both straight sides and curved sides.

Example 26: The medical device of any of examples 1-25, wherein the leadlet body defines a sheet defining a first side and a second side opposite the first side, wherein the first side defines a substantially planar first surface and the second side and defines a substantially planar second surface.

Example 27: The medical device of example 26, wherein the leadlet body is configured position the first surface facing toward the surface of the heart when the leadlet body substantially establishes the preset orientation.

Example 28: The medical device of example 26 or example 27, wherein the electrode is configured to contact the surface of the heart when the first surface contacts the tissue of the heart.

Example 29: The medical device of any of examples 1-28, wherein leadlet body is configured to rotate around the longitudinal axis when the device body rotates around the longitudinal axis and the leadlet body substantially establishes the preset orientation.

Example 30: The medical device of any of examples 1-29 further comprising circuitry mechanically supported by the device body, wherein the electrode is operably connected to the circuitry.

Example 31: The medical device of any of examples 1-30, further comprises a second leadlet attached to the IMD, wherein the second leadlet mechanically supports a second electrode.

Example 32: The medical device of example 31, further comprising circuitry mechanically supported by the device body, wherein the second electrode is operably connected to the circuitry.

Example 33: The medical device of example 31 or example 32, wherein the second electrode is configured to contact a surface of the heart.

Example 34: The medical device of example 31 or 32, wherein the second electrode is configured to penetrate and implant in tissues of heart.

Example 35: A method comprising: establishing a radial displacement between a leadlet distal end of a leadlet and a longitudinal axis of a device body using a shape memory material configured to urge the leadlet body toward a preset orientation relative to the device body, wherein the leadlet body is between a leadlet proximal end and the leadlet distal end, wherein the leadlet proximal end is attached to the device body, and wherein the longitudinal axis extends between a device proximal end of the device body and a device distal end of the device body; and attaching a fixation mechanism to tissue of a heart, wherein the fixation mechanism is attached to the device distal end, wherein the device body is configured to position within the heart, and wherein the leadlet mechanically supports an electrode configured to contact a surface of the heart when the shape memory material urges the leadlet body toward the preset orientation.

Example 36: The method of example 35, further comprising establishing the radial displacement greater than a maximum radial displacement from the longitudinal axis defined by the device body.

Example 37: The method of example 35 or example 36, further comprising extending the leadlet body toward the surface of the heart using the preset orientation.

Example 38: The method of any of examples 35-37, further comprising generating an internal stress within the shape memory material tending to oppose an external force exerted on the leadlet body when the leadlet body substantially establishes the preset orientation.

Example 39: The method of any of examples 35-38, further comprising: penetrating the surface of the heart using the fixation mechanism to attach the fixation mechanism to the tissue of the heart; and contacting the surface of the heart with the electrode when the fixation mechanism attaches to the tissue of the heart and the shape memory material urges the leadlet body toward the preset orientation.

Example 40: The method of any of examples 35-39, further comprising radially displacing the electrode from the longitudinal axis when the leadlet establishes the radial displacement between the leadlet distal end and the longitudinal axis.

Example 41: The method of any of examples 35-40, further comprising extending the leadlet body from the device distal end when the leadlet body substantially establishes the preset orientation.

Example 42: The method of any of examples 35-41, further comprising extending the leadlet body from a portion of the device body proximal to the device distal end when the leadlet body substantially establishes the preset orientation, wherein the device defines a proximal direction from the device distal end to the device proximal end.

Example 43: The method of any of examples 35-42, further comprising radially displacing a conductor from the longitudinal axis when the leadlet establishes the radial displacement between the leadlet distal end and the longitudinal axis, wherein the conductor is electrically connected to the electrode.

Example 44: The method of example 43, further comprising deliver therapy signals to the heart using circuitry of the medical device, wherein the circuitry is electrically connected to the conductor.

Example 45: The method of any of examples 35-44, and further comprising attaching the fixation mechanism to the tissue of the heart by driving a free end of a tine radially outward from the longitudinal axis using a resilient biasing of the tine, wherein the tine includes a fixed end opposite the free end and wherein the tine is mechanically coupled to the device body.

Example 46: The method of example 45, wherein the tine is a first tine, and further comprising: attaching the fixation mechanism to the tissue of the heart by driving a free end of a second tine radially outward from the longitudinal axis using a resilient biasing of the second tine, wherein the second tine includes a fixed end opposite the free end of the second tine, wherein the second tine is mechanically coupled to the device body; and extending the leadlet body between the first tine and the second tine when the leadlet body substantially establishes the preset orientation.

Example 47: The method of any of examples 35-46, wherein the electrode is a first contact electrode, and further comprising: radially displacing a first conductor from the longitudinal axis when the leadlet establishes the radial displacement between the leadlet distal end and the longitudinal axis, wherein the first conductor is electrically connected to the first contact electrode; and radially displacing a second conductor from the longitudinal axis when the leadlet establishes the radial displacement between the leadlet distal end and the longitudinal axis, wherein the second conductor is electrically connected to a second contact electrode.

Example 48: The method of example 47, further comprising at least one of: delivering therapy signals to the heart via the first contact electrode using circuitry of the medical device, wherein the circuitry is electrically connected to the first conductor, or delivering therapy signals to the heart via the second contact electrode using the circuitry of the medical device, wherein the circuitry is electrically connected to the second conductor.

Example 49: The method of any of examples 39-48, further comprising positioning a substantially planar first surface of the leadlet body toward the surface of the heart when the shape memory material urges the leadlet body toward the preset orientation, wherein the leadlet body defines a sheet defining a first side and a second side opposite the first side, wherein the first side defines the first surface and the second side defines a substantially planar second surface.

Example 50: The method of example 49, further comprising contacting the surface of the heart with the electrode when the first surface contacts the tissue of the heart.

Example 51: The method of any of examples 35-50, further comprising deploying the device body from a cup section of a delivery catheter to cause the fixation mechanism to attach to tissue of the heart.

Example 52: The method of any of examples 35-50, further comprising contacting the heart with an additional electrode mechanically supported by a second leadlet extending from the device body.

Various examples of the disclosure have been described. Any combination of the described systems, operations, or functions is contemplated. These and other examples are within the scope of the following claims.

What is claimed is:

1. A medical device comprising:
a device body configured to position within a heart, the device body defining a device proximal end and a device distal end, and the device defining a longitudinal axis extending between the device proximal end and the device distal end;
a fixation mechanism attached to a device distal end, wherein the fixation mechanism is configured to attach to tissue of the heart; and
a leadlet mechanically supporting an electrode,
wherein the leadlet defines a leadlet proximal end, a leadlet distal end, and a leadlet body between the leadlet proximal end and the leadlet distal end,
wherein the leadlet proximal end is attached to the device body,
wherein the leadlet body comprises a shape memory material configured to urge the leadlet body toward a preset orientation relative to the device body, and
wherein the leadlet is configured to define a radial displacement between the leadlet distal end and the longitudinal axis, or an axis parallel to the longitudinal axis, when the shape memory material urges the leadlet body toward the preset orientation.

2. The medical device of claim 1, wherein the electrode is configured to contact a surface of the heart when the fixation mechanism attaches to the tissues of the heart and the shape memory material urges the leadlet body toward the preset orientation.

3. The medical device of claim 1, wherein the shape memory material is configured to generate an internal stress tending to oppose an external force exerted on the leadlet body when the shape memory material urges the leadlet body toward the preset orientation.

4. The medical device of claim 1, wherein the leadlet body defines a curvature between the leadlet proximal end and the leadlet distal end configured to cause the electrode to contact a surface of the heart when the fixation mechanism is attached to the tissues of the heart and the shape memory material urges the leadlet body toward the preset orientation.

5. The medical device of claim 1, wherein the leadlet body defines a facing surface configured to substantially face a surface of the heart when the fixation mechanism is attached to the tissues of the heart and the shape memory material urges the leadlet body toward the preset orientation.

6. The medical device of claim 1, wherein the device body defines a maximum radial displacement from the longitudinal axis, and wherein the radial displacement between the leadlet distal end and the longitudinal axis is greater than the maximum device radial displacement.

7. The medical device of claim 1, wherein the leadlet is configured to radially displace the electrode from the longitudinal axis or the axis parallel to the longitudinal axis when the leadlet defines the radial displacement between the leadlet distal end and the longitudinal axis.

8. The medical device of claim 1, further comprising a conductor mechanically supported by the leadlet body,
wherein the conductor is electrically connected to the electrode, and
wherein the leadlet is configured to radially displace the conductor from the longitudinal axis or the axis parallel to the longitudinal axis when the leadlet defines the radial displacement between the leadlet distal end and the longitudinal axis.

9. The medical device of claim 8, further comprising circuitry configured to deliver therapy signals to the heart using the electrode, wherein the conductor is electrically connected to the circuitry.

10. The medical device of claim 1, wherein the shape memory material comprises a polymer.

11. The medical device of claim 1, wherein the fixation mechanism includes one or more tines, wherein a tine includes a fixed end and a free end, wherein the fixed end is mechanically coupled to the device body, and wherein the tine is biased to drive the free end radially outward from the longitudinal axis.

12. The medical device of claim 11, wherein:
the one or more tines includes a first tine and a second tine,
the leadlet is secured to the device distal end, and
the leadlet body is configured to pass between the first tine and the second tine when the leadlet body substantially establishes the preset orientation.

13. The medical device of claim 1, wherein the electrode is a first contact electrode, and further comprising:
a first conductor mechanically supported by the leadlet body, wherein the first conductor is electrically connected to the first contact electrode and electrically connected to circuitry of the medical device;
a second contact electrode mechanically supported by the leadlet body; and
a second conductor mechanically supported by the leadlet body, wherein the second conductor is electrically connected to the second contact electrode and electrically connected to the circuitry of the medical device,
wherein the circuitry of the medical device is configured to deliver therapy signals to the heart using at least one of the first contact electrode or the second contact electrode.

14. The medical device of claim 1, wherein the leadlet body defines a sheet defining a first side and a second side opposite the first side, wherein the first side defines a substantially planar first surface and the second side and defines a substantially planar second surface, wherein the leadlet body is configured position the first surface facing toward the surface of the heart when the fixation mechanism attaches to the tissue of the heart and the leadlet body substantially establishes the preset orientation.

15. The medical device of claim 1, further comprising a distal electrode extending from a distal portion of the device body, wherein the distal portion includes the device distal end, and wherein the distal electrode is configured to flexibly maintain contact with wall tissue of the heart when the fixation mechanism is attached to tissue of the heart.

16. A medical device comprising:
a device body configured to position within a heart, the device body defining a device proximal end and a device distal end, and the device defining a longitudinal axis extending between the device proximal end and the device distal end;
a fixation mechanism attached to a device distal end, wherein the fixation mechanism is configured to attach to tissue of the heart; and
a leadlet mechanically supporting an electrode,
wherein the leadlet defines a leadlet proximal end, a leadlet distal end, and a leadlet body between the leadlet proximal end and the leadlet distal end,
wherein the leadlet proximal end is attached to the device body,
wherein the leadlet body comprises a shape memory material configured to urge the leadlet body toward a preset orientation relative to the device body,
wherein the leadlet is configured to define a radial displacement between the leadlet distal end and the longitudinal axis, or an axis parallel to the longitudinal axis, when the shape memory material urges the leadlet body toward the preset orientation,
wherein the electrode is configured to contact a surface of the heart when the fixation mechanism attaches to the tissues of the heart and the shape memory material urges the leadlet body toward the preset orientation, and
wherein the shape memory material is configured to generate an internal stress tending to oppose an external force exerted on the leadlet body when the shape memory material urges the leadlet body toward the preset orientation.

17. The medical device of claim 16, wherein the leadlet is configured to radially displace the electrode from the longitudinal axis or the axis parallel to the longitudinal axis when the leadlet defines the radial displacement between the leadlet distal end and the longitudinal axis.

18. The medical device of claim 16, further comprising:
a conductor mechanically supported by the leadlet body,
wherein the conductor is electrically connected to the electrode, and
wherein the leadlet is configured to radially displace the conductor from the longitudinal axis or the axis parallel to the longitudinal axis when the leadlet defines the radial displacement between the leadlet distal end and the longitudinal axis; and circuitry configured to deliver therapy signals to the heart using the electrode,
wherein the conductor is electrically connected to the circuitry.

19. A method comprising:
establishing a radial displacement between a leadlet distal end of a leadlet and a longitudinal axis of a device body or an axis parallel to the longitudinal axis using a shape memory material configured to urge the leadlet body toward a preset orientation relative to the device body, wherein the leadlet body is between a leadlet proximal end and the leadlet distal end, wherein the leadlet proximal end is attached to the device body, and wherein the longitudinal axis extends between a device proximal end of the device body and a device distal end of the device body; and
attaching a fixation mechanism to tissue of a heart, wherein the fixation mechanism is attached to the device distal end, wherein the device body is configured to position within the heart, and wherein the leadlet mechanically supports an electrode configured to contact a surface of the heart when the shape memory material urges the leadlet body toward the preset orientation.

20. The method of claim 19, further comprising:
penetrating the surface of the heart using the fixation mechanism to attach the fixation mechanism to the tissue of the heart; and
contacting the surface of the heart with the electrode when the fixation mechanism attaches to the tissue of the heart and the shape memory material urges the leadlet body toward the preset orientation.
